(19) United States
(12) Patent Application Publication
(10) Pub. No.: US 2026/0315275 A1
(43) Pub. Date: Oct. 15, 2026

(54) CONVERTIBLE DIAPHRAGM GAS PUMP SYSTEM

(71) Applicant: ALCON INC., Fribourg (CH)

(72) Inventor: Brian T. Chiem, San Gabriel, CA (US)

(21) Appl. No.: 19/467,566

(22) Filed: Feb. 2, 2026

Related U.S. Application Data
(60) Provisional application No. 63/753,678, filed on Feb. 4, 2025.

(51) Int. Cl. A61F 9/007; A61M 1/00

(52) U.S. Cl. CPC A61F 9/00736; A61M 1/77; A61M 2210/0612

(57) ABSTRACT

The present disclosure relates to a vacuum system for an ophthalmic surgical system. The vacuum system includes a vacuum accumulator, a patient interface, and a vacuum generation system. The patient interface is pneumatically coupled to the vacuum accumulator and engageable with patient tissue responsive to application of vacuum pressure from the vacuum accumulator through the patient interface to the patient tissue. The vacuum generation system is coupled to the vacuum accumulator and includes multiple diaphragm gas pump heads and one or more solenoid valves. The multiple diaphragm gas pump heads include at least a first head and a second head. The one or more solenoid valves are coupled to the first and second heads and are configured to selectively pneumatically couple the first head and the second head in parallel or series. The system may further include a pressure accumulator and may be selectively switched between vacuum or pressure accumulation.

### CROSS-REFERENCE TO RELATED APPLICATION

This application claims priority to U.S. Provisional Patent Application No. 63/753,678, filed on Feb. 4, 2025, the disclosure of which is incorporated herein by reference in its entirety.

### BACKGROUND

Diaphragm gas pumps are commonly used in medical devices to generate vacuum pressure. These pumps typically operate in either a parallel or series configuration. The parallel configuration offers higher flow rate but lower maximum vacuum pressure, while the series configuration offers higher vacuum pressure but lower flow rate. Existing systems are generally fixed in one configuration, limiting their flexibility and performance across different operating conditions.

### SUMMARY

The present disclosure relates to a vacuum system for an ophthalmic surgical system. The vacuum system includes a vacuum accumulator, a patient interface, and a vacuum generation system. The vacuum system may further include a pressure controller. The patient interface is pneumatically coupled to the vacuum accumulator, e.g., indirectly via one or more intervening components, such as the pressure controller. The patient interface is engageable with patient tissue responsive to application of vacuum pressure from the vacuum accumulator through the patient interface to the patient tissue. The vacuum generation system is coupled to the vacuum accumulator and includes a multi-diaphragm gas pump and one or more solenoid valves. The multi-head diaphragm gas pump includes at least a first head and a second head. The one or more solenoid valves are coupled to the first head and the second head and are configured to selectively pneumatically couple the first and second heads in parallel or series.

### BRIEF DESCRIPTION OF THE DRAWINGS

The present disclosure relates to systems and methods for performing registration of an eye based on transition edges within the eye, wherein:

FIG. 1 is a block diagram of an example ophthalmic surgical system that may be used with and/or implement one or more embodiments of the present disclosure related to a convertible diaphragm gas pump system;

FIG. 2 is a schematic diagram of an example vacuum system that may be included in the ophthalmic surgical system of FIG. 1;

FIG. 3 is a schematic diagram of an example vacuum generation system that may be included in the vacuum system of FIG. 2;

FIGS. 4A-4D are schematic diagrams of various example vacuum generation systems selectively operable in a parallel or series mode;

FIG. 4E is a schematic diagram of two types of 5/3 solenoid valves that may be implemented in any of the vacuum systems herein;

FIGS. 5A-5D are schematic diagrams of various example vacuum generation systems selectively operable to generate vacuum pressure or positive pressure;

FIGS. 6A-6J are schematic diagrams of various example vacuum systems selectively operable in a parallel or series mode and selectively operable to generate vacuum pressure or positive pressure;

FIG. 7 is a flowchart of an example method to operate a vacuum system for an ophthalmic surgical system;

FIG. 8 illustrates an example of how to determine whether to operate a multi-head diaphragm gas pump in parallel or series based on measured barometric pressure and capabilities of the multi-head diaphragm gas pump;

FIG. 9 is a flowchart of another example method to operate a vacuum system for an ophthalmic surgical system; and

FIG. 10 is a block diagram of an example computing system suitable for use in implementing some embodiments of the present disclosure.

### DETAILED DESCRIPTION

Diaphragm gas pumps are a type of positive displacement pump. A pump head typically consists of a motor that rotates to move a membrane or diaphragm in reciprocating motion which causes gases to move, and two check-valves (e.g., devices that allow gas to move in only one direction). The effect of this mechanism causes gas to enter through an inlet check valve and into a chamber on the diaphragm's down-stroke, and the gas to exit through the outlet check valve during the diaphragm's up-stroke.

When the inlet of the pump is connected to a vacuum accumulator or reservoir or other closed volume or chamber, and the outlet is connected to open air, gas molecules are evacuated from the accumulator and moved into open air, thus causing vacuum pressure to build inside the reservoir. However, as the vacuum pressure builds, it becomes harder and harder to evacuate the remaining gas molecules so the pump typically has a maximum vacuum pressure it can achieve based on its design and quality of manufacturing.

Many diaphragm gas pump manufacturers offer a double-headed version which consists of two pairs of inlet ports and outlet ports. They are typically used in parallel or series. In the parallel configuration, the two inlet ports are coupled to the vacuum accumulator and the two outlet ports are coupled to open air (or to a positive pressure accumulator or reservoir). In the series configuration, the inlet port of a first pump head is connected to the vacuum accumulator, the outlet port of the first pump head is coupled to the inlet port of a second pump head, and the outlet port of the second pump head is coupled to open air (or to the positive pressure accumulator or reservoir). The parallel configuration typically offers higher flow rate but lower maximum achievable vacuum pressure. The series configuration typically offers higher maximum achievable vacuum pressure but lower flow rate.

Existing multi-head diaphragm gas pumps are typically configured in series or parallel only or, to the extent they are configurable, must be configured in manufacturing or service depending on an intended destination of the multi-head diaphragm gas pump. Thus, existing multi-head diaphragm gas pumps are typically stuck with the disadvantages of their respective configuration and cannot be easily reconfigured (if at all) even if warranted under the circumstances.

Some alternatives to a single double-headed diaphragm gas pump include two double-headed diaphragm gas pumps (first configured in parallel, second configured in series) with a solenoid valve to switch/select the desired configuration and two single-head diaphragm gas pumps used in parallel or series. The alternative of two double-headed diaphragm gas pumps disadvantageously requires multiple pumps, increasing cost. The alternative of two single-head diaphragm gas pumps used in parallel or series has the same problems as a single double-headed diaphragm gas pump.

An example ophthalmic surgical system, LENSX offered by ALCON, uses a diaphragm gas pump to generate vacuum pressure in a vacuum accumulator, which is then used to provide suction during surgery to attach a patient interface of the system to the patient's eye. The pump may use a parallel configuration by default. Above a certain altitude, however, and depending on the specific diaphragm gas pump used and its configuration, its achievable maximum vacuum may be reduced because of lower atmospheric pressure, which is a physical limitation of such pumps. To maximize the altitude at which such products can maintain normal operation, the diaphragm gas pump may be carefully selected for higher maximum possible vacuum and/or configured in the series configuration. However, this comes at the performance cost of lower flow rate (in the series configuration) which means the vacuum accumulator may take a longer time to reach the desired vacuum pressure, requiring longer pump on-time and reduced lifetime, and generating pump noise when on. Some embodiments herein may allow a control system to switch between the parallel configuration to evacuate at a higher flow rate and lower maximum achievable vacuum, and the series configuration to evacuate at a lower flow rate and a higher maximum achievable vacuum. The decision on which mode to operate in (parallel versus series) may be decided when the system is installed at a surgical center and may be set in software.

Other vacuum systems use a venturi vacuum ejector or oil-sealed vacuum pumps instead of multi-head diaphragm gas pumps. Venturi vacuum ejectors require compressed gas (e.g., air) at a high flow rate to achieve maximum vacuum. Typically, this requires a supply of compressed gas from a source (e.g., compressed gas tanks or a compressor). The compressed gas supplies and/or compressed gas tanks are additional equipment a surgical center would require to implement venturi vacuum ejectors. Compressors create additional device complexity and noise during ophthalmic surgical procedures.

Oil sealed vacuum pumps require maintenance, oil refills, and create additional noise during operation. The noise created by a compressor or oil sealed vacuum pumps can be an irritant to patients during ophthalmic surgical procedures.

Accordingly, some embodiments herein relate to a system that includes a multi-head diaphragm gas pump with one or more solenoid valves to convert between parallel and series configurations of the heads of the pump. More generally, the system includes multiple diaphragm gas pump heads (which may be different heads of the same diaphragm gas pump or may be multiple single-head diaphragm gas pumps) with one or more solenoid valves to convert between parallel and series configurations of the heads. For simplicity in the discussion that follows, all pumps are described as multi-head diaphragm gas pumps. However, those skilled in the art with the benefit of the present disclosure will understand that that the pumps may instead include multiple single-head diaphragm gas pumps.

Some embodiments further include a control system that may include a computer, microcontroller, central processor unit (CPU), or the like, and software to selectively operate the system in parallel (i.e., with the heads of the pump configured in parallel) or series (i.e., with the heads of the pump configured in series).

In some embodiments, the configuration (in parallel or series) may be a software setting initially selected by a user, field service engineer, manufacturing engineer, or other individual. If a different mode is desired later, the software setting may be changed easily in software. As such, there may be no need to open the system and mechanically change the configuration as it may be handled completely with the switch of the software setting. Such a setting may be a more rigid change (e.g., requiring powering down and powering up the device to effect the change in mode of operation) or a more fluid change (e.g., being changed on-the-fly during operation). Advantageously, diaphragm gas pumps may remove the need for external or internal compressed air supplies compared to a Venturi vacuum ejector. Further, diaphragm gas pumps do not need oil refills and have less frequent needs for maintenance compared to oil-sealed vacuum pumps.

In general, the one or more solenoid valves may include multiple ports with two or more positions where different ports are connected across the valve in different positions. Each valve may be energized to move it to one position and one set of interconnections between ports or de-energized to move it to another position and another different set of interconnections between ports. In some embodiments, a solenoid valve may include two (or more) energized states corresponding to different energization levels where different ports are connected across the valve in each of three (or more) different positions corresponding to de-energized and each of the two (or more) energized states. Each solenoid valve may be energized (e.g., to a given energization level) or de-energized based on the software setting.

Some embodiments further include a barometric pressure sensor to measure barometric (or atmospheric) pressure at a location where the system will be used to determine if parallel mode will be sufficient to reach a desired vacuum pressure. If so, the system may be operated in parallel mode. If not, the system may be operated in series mode. Barometric pressure generally decreases as altitude increases. The maximum achievable vacuum pressure of the system may depend on the barometric pressure and may typically decrease as the barometric pressure decreases. Thus, while the system in parallel mode may be able to achieve the desired vacuum pressure at a first barometric pressure of a first location, it may be unable in parallel mode to achieve the desired vacuum pressure at a second barometric pressure of a second higher altitude location and so may instead be set to operate in series mode. The barometric pressure sensor, combined with the easily reconfigurable operating mode (e.g., parallel or series) of the system, may allow the operating mode of the system to, e.g., be set on site in dependence on the barometric pressure without a priori knowledge of the usage location or manual mechanical reconfiguration.

Alternatively or additionally, some embodiments further include a pressure sensor to measure vacuum pressure in the vacuum accumulator. The diaphragm gas pump may initially and/or by default operate in a parallel mode while the pressure sensor senses the pressure in the vacuum accumulator. A control system may monitor pressure measurements generated by the pressure sensor and switch to the series mode when the vacuum pressure nears the maximum achievable vacuum pressure in the parallel mode. For example, the control system may determine to switch to the series mode when the measured vacuum pressure reaches a threshold (that may have been calibrated previously) and/or when the measured vacuum pressure stabilizes (e.g., at or near a maximum achievable value). By operating initially in the parallel mode before switching to the series mode, the system may minimize or otherwise decrease the amount of time to reach the desired vacuum pressure (and thereby reduce the amount of time a patient is subject to noise and discomfort during the ophthalmic surgical procedure), while still being able to achieve the desired vacuum pressure in the series mode that may not be achievable in the parallel mode.

Alternatively or additionally, some embodiments include one or more other solenoid valves coupled between the diaphragm gas pump and each of the vacuum accumulator, open air, and a pressure accumulator to selectively operate the system to generate vacuum pressure or positive pressure. The one or more other solenoid valves may couple the inlet ports of the pump to the vacuum accumulator and the outlet ports to open air to generate vacuum pressure in the vacuum accumulator. On the other hand, the one or more other solenoid valves may couple the inlet ports of the pump to open air and the outlet ports to the pressure accumulator to generate positive pressure in the pressure accumulator. Similar to the parallel and series operating modes, the vacuum generation or pressure generation modes may be set by a software setting and may be controlled electronically. Further, the various embodiments may be combined such that a system may be both selectively operable in parallel or series and selectively operable to generate vacuum pressure or positive pressure.

The embodiments of the present disclosure will be explained with reference to the accompanying figures. It is to be understood that the figures are diagrammatic and schematic representations of such example embodiments, and are not limiting, nor are they necessarily drawn to scale. In the figures, features with like numbers indicate like structure and function unless described otherwise. Further, one or more of the figures and accompanying descriptions are given with respect to ophthalmic surgical systems. However, such uses are not meant to be limiting such that the devices described may be used in any number of different contexts and applications where it may be helpful or applicable.

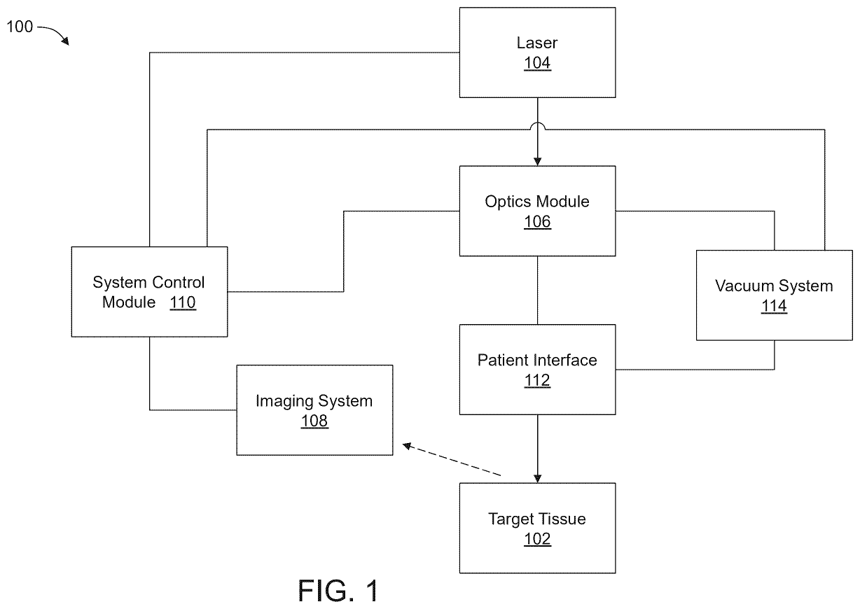

FIG. 1 is a block diagram of an example ophthalmic surgical system 100 (“system 100”) that may be used with and/or implement one or more embodiments of the present disclosure related to a convertible diaphragm gas pump system. In general, the system 100 may be configured to perform one or more ophthalmic treatment operations with respect to performance of a procedure corresponding to a target tissue 102, which may include a portion of an eye in some embodiments. In some embodiments, the system 100 may include one or more of a laser 104, an optics module 106, an imaging system 108, a system control module 110, a patient interface 112, and a vacuum system 114.

The laser 104 may include any suitable system, apparatus, or device, configured to generate one or more laser beams that may be used to perform ophthalmic operations or tasks with respect to the target tissue 102. In some embodiments, the laser 104 may include multiple lasers that each generate an individual laser beam. Additionally or alternatively, the laser 104 may include a single laser that is configured to generate a single beam or multiple beams.

In some embodiments, the laser 104 may be configured to generate a pulsed laser beam that is pulsed at a high repetition rate at a pulse repetition rate of thousands of shots per second or higher with relatively low energy per pulse. For example, in some embodiments, the laser 104 may be a femtosecond laser that emits ultra-short pulses of light (e.g., on the order of 10\hat{ }-15 seconds). Such a laser may be operated to use a relatively low energy per pulse to localize the tissue effect within the target tissue 102 that may be caused by laser-induced photodisruption by the beam generated by the laser 104.

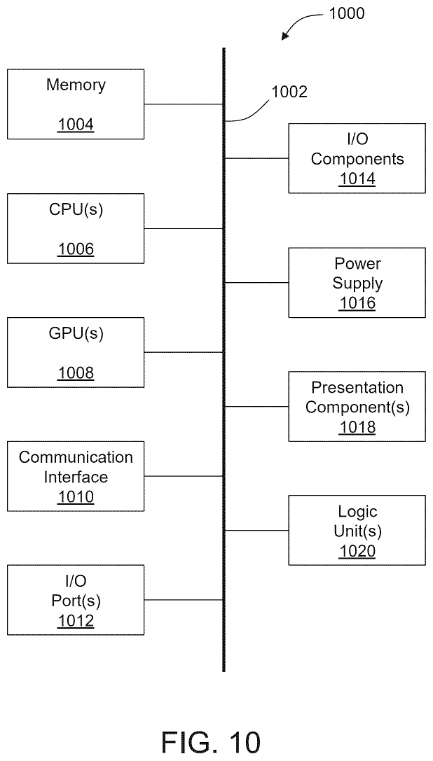

The optics module 106 may include any suitable system, apparatus, or device that may be configured to focus and direct the laser beam to the target tissue 102. For example, in some embodiments, the optics module 106 may include one or more lenses and/or one or more reflectors (e.g., mirrors). Additionally or alternatively, in some embodiments, the optics module 106 may include one or more actuators that may be configured to adjust the focusing and/or the beam direction in response to a beam control signal that may be received from the system control module 110. In these and other embodiments, the one or more actuators may be adjusted in response to a user input via any suitable user interface, such as discussed with respect to a computing system of FIG. 10. For example, in some embodiments, the user interface may include a touch screen, mouse, keyboard, joystick, foot pedal, game pad, game controller, etc., that may be used to provide commands for movement of the laser beam (e.g., via the actuators).

The imaging system 108 may include any suitable system, apparatus, or device, that may be configured to obtain one or more images of the eye corresponding to the target tissue 102. For example, in some embodiments, the imaging system 108 may collect reflected or scattered light or sound from the target tissue 102 to capture image data corresponding to the target tissue 102.

The imaging system 108 may include one or more different types of devices and/or systems configured to capture various different types of images of the eye as corresponding to the target tissue 102. For example, in some embodiments, the imaging system 108 may include a camera configured to capture one or more camera images of the eye. In these and other embodiments, the imaging system may include an ultrasound imaging device configured to capture ultrasound images of the eye. Additionally or alternatively, the imaging system 108 may include an optical coherent tomography (OCT) device configured to capture OCT images of the eye or other imaging device(s), or other imaging system or device.

The patient interface 112 may include a mount that is configured to engage with the target tissue 102 to hold the target tissue 102 in position (or to hold the patient interface 112 in position with respect to the target tissue 102) during performance of the ophthalmic procedure. In these and other embodiments, the patient interface 112 may be configured to allow the laser beam to pass therethrough to allow for performance of the procedure via the laser beam.

The vacuum system 114 may include various systems and/or devices to, e.g., selectively generate a vacuum (or more generally, negative pressure) for use in the system 100. For example, the patient interface 112 may include a suction ring with a pneumatic port that may be coupled (e.g., through a pneumatic line) to the vacuum system 114 which may apply a vacuum to the suction ring so the suction ring may engage the target tissue 102 during performance of the ophthalmic procedure. Alternatively or additionally, a portion of the optics module 106, such as a laser head, may include a pneumatic port that may be coupled to the vacuum system 114 which may apply a vacuum to the portion to dock the portion of the optics module 106 to the patient interface 112 during performance of the ophthalmic procedure. In these and other embodiments, the vacuum system 114 may be selectively operable in a parallel mode or a series mode and/or in a vacuum mode or a positive pressure mode.

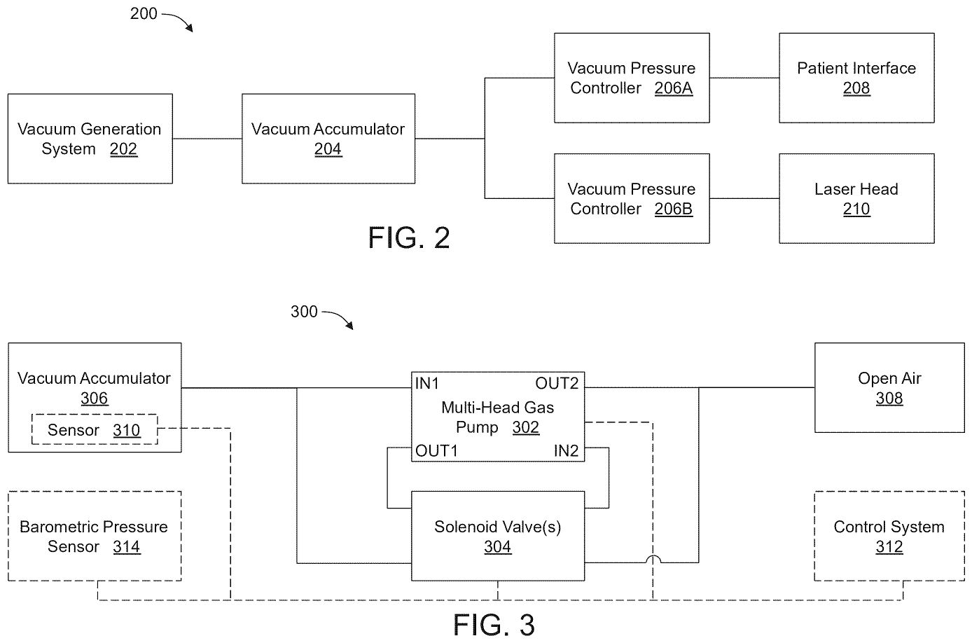

FIG. 2 is a schematic diagram of an example vacuum system 200 that may be included in the system 100 of FIG. 1, arranged in accordance with at least one embodiment herein. The vacuum system 200 may include, be included in, or otherwise correspond to the vacuum system 114 of FIG. 1 or other vacuum systems herein. The vacuum system 200 may include a vacuum generation system 202, a vacuum accumulator 204, and one or more vacuum pressure controllers 206A, 206B (hereinafter generically “pressure controller 206” or collective “pressure controllers 206”). The pressure controller 206A may be coupled to a patient interface 208 that may include, be included in, or otherwise correspond to the patient interface 112 of FIG. 1. The pressure controller 206B may be coupled to a laser head 210 that may be included in the optics module 106 of FIG. 1. The various components in FIG. 2 are connected by lines that may represent pneumatic connections.

The vacuum generation system 202 is configured to generate vacuum pressure. The term “vacuum” as used herein should be broadly construed to refer to lower relative, but not necessarily zero, pressure, i.e., at least partial vacuum in which gaseous pressure is less than atmospheric pressure. In some embodiments, the vacuum generation system 202 may include one or more gas pumps and one or more solenoid valves. In these and other embodiments, each of the gas pumps may include a multi-head diaphragm gas pump such as a two-head diaphragm gas pump, a four-head diaphragm gas pump, or other multi-head diaphragm gas pump, or two or more single-head diaphragm gas pumps. Each of the solenoid valves may include multiple ports with multiple positions, such as a 3/2 (i.e., 3 ports and 2 positions) solenoid valve, a 4/2 solenoid valve, a 5/2 solenoid valve, a 5/3 solenoid valve, or other suitable solenoid valve.

The vacuum accumulator 204 is coupled to the vacuum generation system 202 and is configured to store vacuum pressure.

Each of the pressure controllers 206 is coupled to the vacuum accumulator 204 and is configured to regulate the amount of vacuum applied, from the vacuum accumulator 204, at the corresponding patient interface 208 or laser head 210.

In operation, the vacuum generation system 202 generates vacuum pressure which is stored in the vacuum accumulator 204. In general, vacuum pressure may be generated by the vacuum generation system 202, and more particularly the pump of the vacuum generation system 202, pumping air out of the vacuum accumulator 204. The vacuum pressure stored in the vacuum accumulator 204 may then be distributed through either or both of two parallel paths that respectively end at the patient interface 208 and/or the laser head 210. By applying vacuum pressure at the patient interface 208, the patient interface 208 may engage a target tissue (e.g., the target tissue 102 of FIG. 1), such as an eye of a patient, by suctioning to the target tissue. The vacuum pressure applied at the laser head 210 may facilitate the laser head 210 docking to the patient interface 208 by suctioning to the patient interface 208. The parallel configuration of the paths allows independent control of vacuum pressure (by the pressure controllers 206) for the patient interface suction and the laser head docking from a single vacuum source (e.g., the vacuum accumulator 204). The pressure controllers 206 may allow precise regulation of the vacuum levels provided to each of the patient interface 208 and the laser head 210.

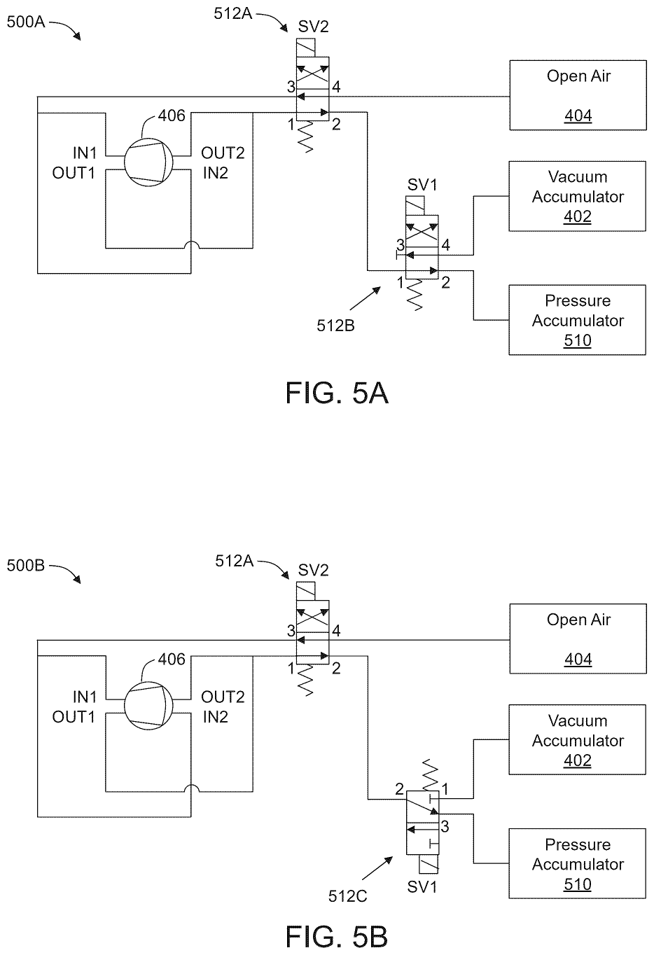

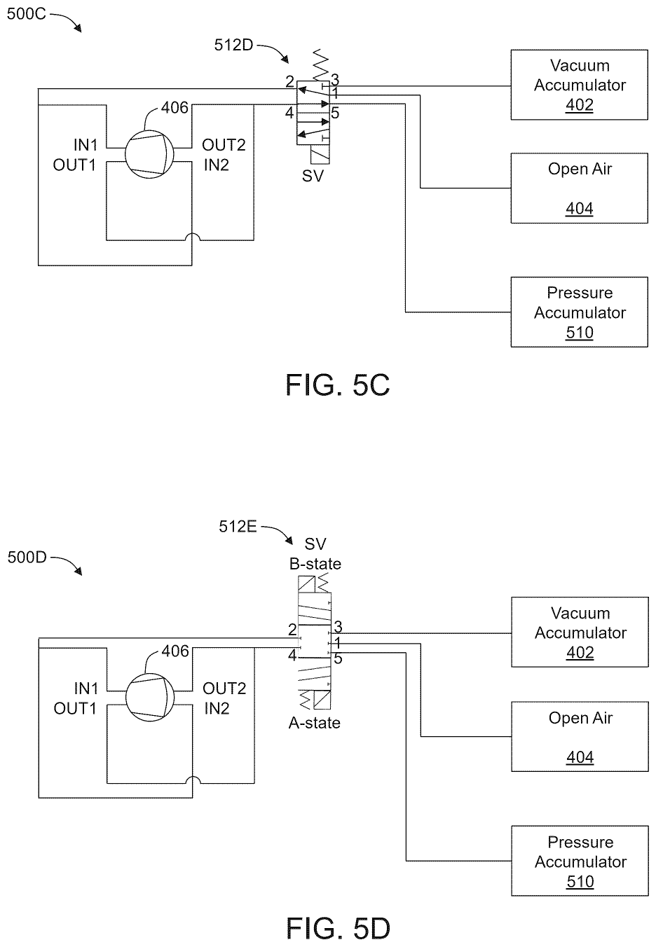

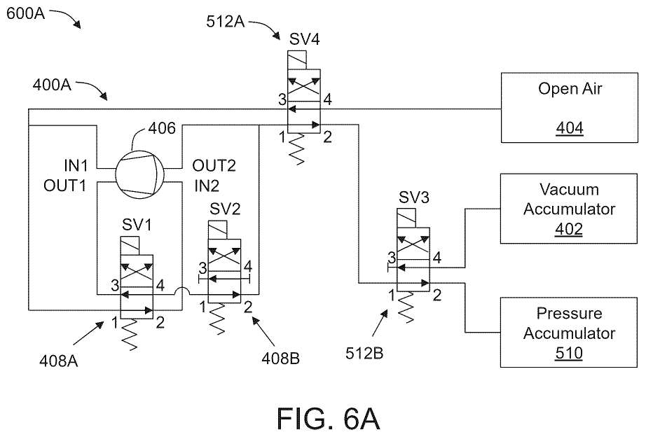

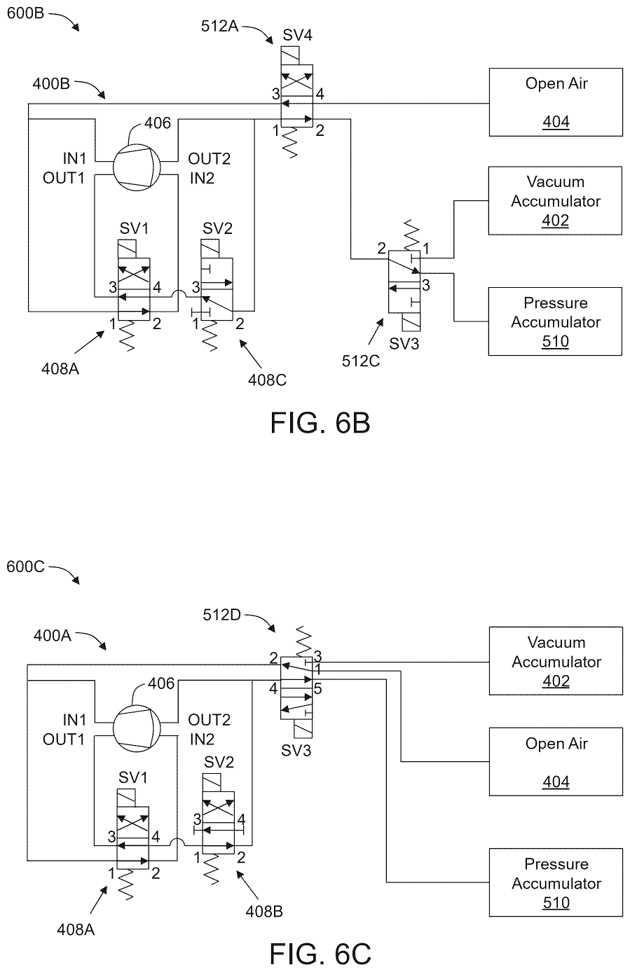

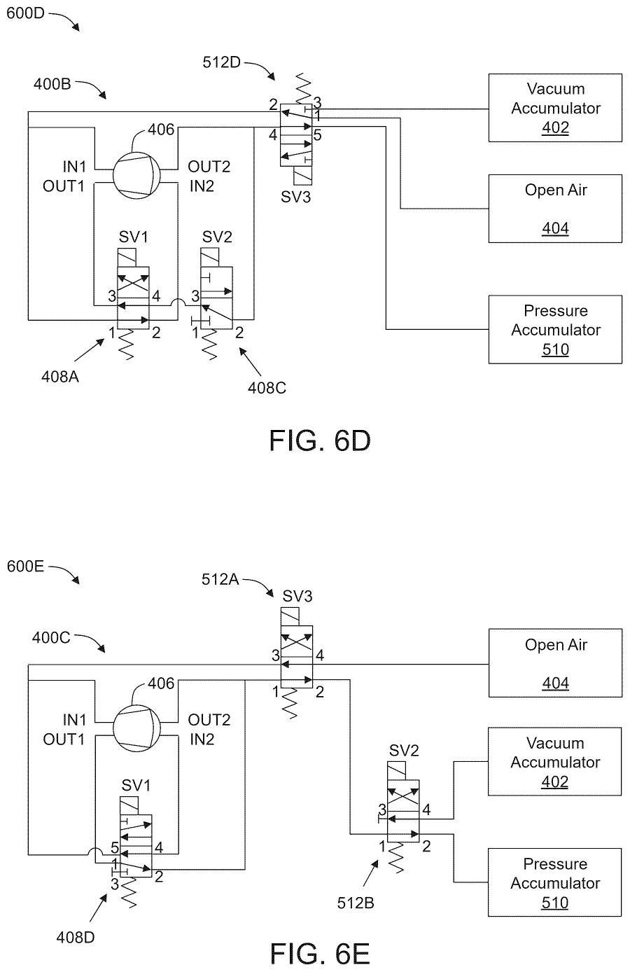

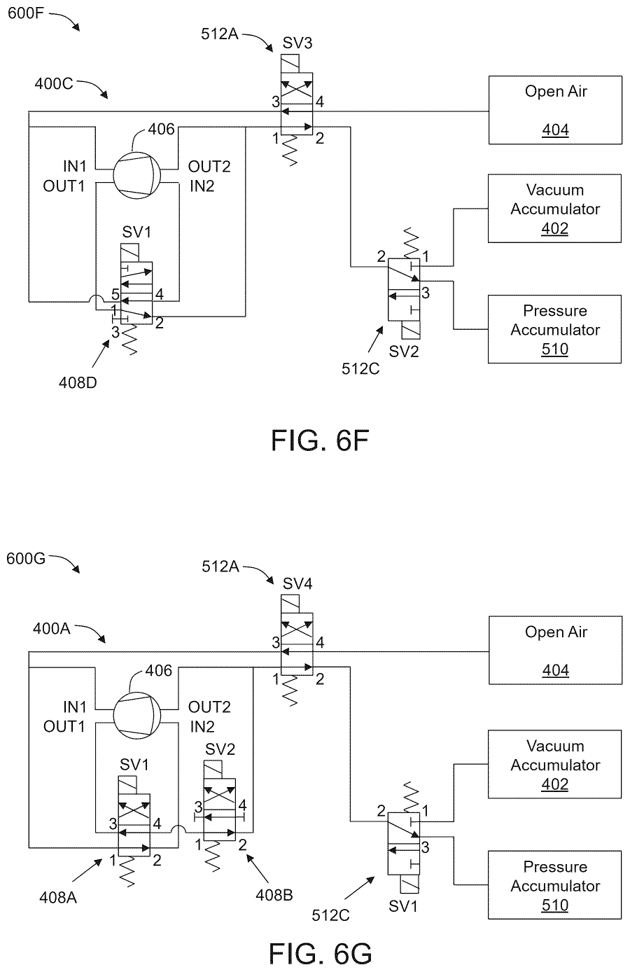

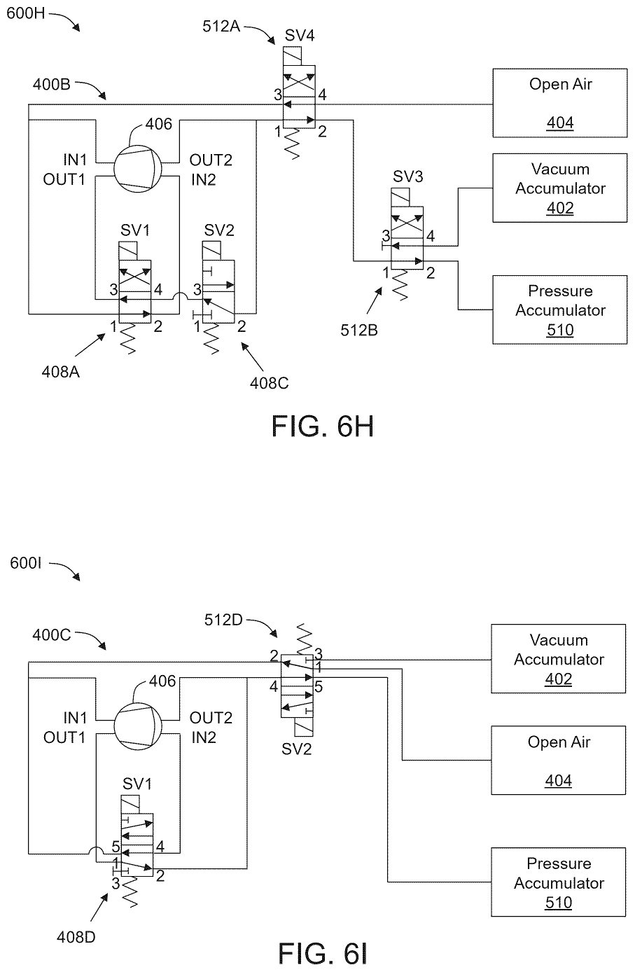

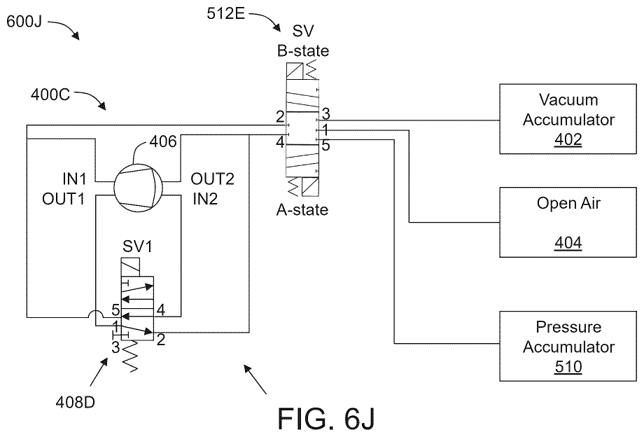

Modifications, additions, or omissions may be made to the vacuum system 200 without departing from the scope of the present disclosure. For example, in some embodiments, the vacuum generation system 202 may be further coupled to open air (directly or via one or more intervening components such as a muffler or filter) and a pressure accumulator such as described with respect to FIGS. 5A-6J. The vacuum generation system 202 may be coupled to the vacuum accumulator 204 and open air to pump air from the vacuum accumulator 204 and release it to open air (and thereby increase vacuum pressure within the vacuum accumulator). Alternatively, the vacuum generation system 202 may be coupled to the pressure accumulator and open air to pump air from the open air into the pressure accumulator (and thereby increase positive pressure within the pressure accumulator). Alternatively or additionally, the vacuum system 200 may include one or more solenoid valves coupled between the vacuum generation system 202 and each of the vacuum accumulator, the pressure accumulator, and open air to selectively pneumatically couple the vacuum generation system 202 to the vacuum accumulator (and open air) or the pressure accumulator (and open air).

As another example, the vacuum system 200 and/or one or more of its components (such as the vacuum generation system 202) may be coupled to a control system that may include a computing system. The control system may include, be included in, or correspond to, e.g., the system control module 110 of FIG. 1 and/or other systems or devices herein. The control system may electronically control one or more components of the vacuum system 200.

FIG. 3 is a schematic diagram of an example vacuum generation system 300 that may be included in the vacuum system 200 of FIG. 2, arranged in accordance with at least one embodiment herein. The vacuum generation system 300 may include, be included in, or otherwise correspond to the vacuum generation system 202 of FIG. 2 or other vacuum generation systems herein. The vacuum generation system 300 includes a multi-head diaphragm gas pump 302 (hereinafter “pump 302”) and one or more solenoid valves 304 (hereinafter generically “SV 304” or collectively “SVs 304”). The vacuum generation system 300 may be part of a vacuum system (such as the vacuum system 200 of FIG. 2) that may further include, e.g., a vacuum accumulator 306 and open air 308 coupled to the vacuum generation system 300. The vacuum accumulator 306 may include, be included in, or otherwise correspond to the vacuum accumulator 204 of FIG. 2 or other vacuum accumulators herein. The vacuum generation system 300 may be coupled to open air 308 through one or more intervening parts, such as a muffler or filter. Various components in FIG. 3 are connected by solid lines that may represent pneumatic connections.

The pump 302 may include a two-head diaphragm gas pump, a four-head diaphragm gas pump, or a multi-head diaphragm gas pump with other number of heads in excess of two. Further, the pump 302 includes at least a first head and a second head, each including an inlet port and an outlet port. For example, as illustrated in FIG. 3, the first head of the pump 302 includes a first inlet port IN1 and a first outlet port OUT1 while the second head of the pump 302 includes a second inlet port IN2 and a second outlet port OUT2. The pump 302 may further include one or more check valves that permit one-way gas flow within each head, e.g., from IN1 to OUT1 and from IN2 to OUT2. As illustrated, IN1 of the first head of the pump 302 is pneumatically coupled to the vacuum accumulator 306 and OUT2 of the second head of the pump 302 is pneumatically coupled to open air 308. Although the pump 302 is described as a multi-head diaphragm gas pump, the pump 302 may instead include two or more single-head diaphragm gas pumps. When implemented as, e.g., two single-head diaphragm gas pumps, a first one of the single-head diaphragm gas pumps may include IN1 and OUT1 while a second one of the single-head diaphragm gas pumps may include IN2 and OUT2.

The one or more SVs 304 may include one or more of a 3/2 SV, a 4/2 SV, a 5/2 SV, a 5/3 SV, or any other suitable SV(s). The one or more SVs 304 are generally configured to selectively pneumatically couple the heads of the pump 302 (or the heads of multiple single-head pumps) in parallel or series. For example, in a parallel mode, the one or more SVs 304 pneumatically couple the vacuum accumulator 306 to IN2 and open air 308 to OUT1 such that the first and second heads are pneumatically coupled in parallel since IN1 is already pneumatically coupled to the vacuum accumulator 306 (similar to IN2) and OUT2 is already pneumatically coupled to open air 308 (similar to OUT1). In a series mode, the one or more SVs 304 pneumatically couple OUT1 to IN2 such that the first and second heads are pneumatically coupled in series since IN1 is already pneumatically coupled to the vacuum accumulator 306 and OUT2 is already pneumatically coupled to open air 308.

In some embodiments, the vacuum system of FIG. 3 may further include one or more of a pressure sensor 310, a control system 312, and/or a barometric pressure sensor 314. Each of these components is depicted in dashed lines as an indication that it is optional in the example of FIG. 3.

The pressure sensor 310 may be configured to measure vacuum pressure in the vacuum accumulator 306. The pressure sensor 310 may be disposed in and/or coupled to the vacuum accumulator 306. The pressure sensor 310 may provide its measurements to, e.g., the control system 312.

The barometric pressure sensor 314 may be configured to measure barometric pressure at a usage location of the vacuum generation system 300. The usage location refers to a location at which the vacuum generation system 300 is being or will be used. Barometric pressure may be indicative of altitude. The vacuum generation system 300 may have different capabilities at different altitudes or barometric pressures. For example, a maximum vacuum pressure achievable by the vacuum generation system 300 in each of the parallel mode and the series mode may be different at one barometric pressure or altitude than at another barometric pressure or altitude. Barometric pressure measurements may be used to determine whether to configure the vacuum generation system 300 in parallel mode or series mode. For example, if the maximum achievable vacuum pressure of the vacuum generation system 300 in parallel mode under the barometric pressure measurement at the usage location is less than a target vacuum pressure, the vacuum generation system 300 may be configured in series mode to ensure the vacuum generation system 300 may achieve the target vacuum pressure during use. The barometric pressure sensor 314 may provide its measurements to, e.g., the control system 312.

The control system 312 may include, be included in, or correspond to other control systems or devices herein, such as the system control module 110 of FIG. 1 and/or other systems or devices herein. The control system 312 may be communicatively coupled to one or more components in the vacuum system of FIG. 3 to send or receive, e.g., measurements, commands, or the like. The communicative coupling between the control system 312 and various components is depicted in FIG. 3 by dashed lines. In some embodiments, the control system 312 may receive vacuum pressure measurements from the pressure sensor 310 and/or barometric pressure measurements from the barometric pressure sensor 314. Alternatively or additionally, the control system 312 may electronically control the one or more SVs 304 to energize or de-energize (and thereby switch between the parallel and series mode) or the pump 302 to turn on or off or control pump speed.

Modifications, additions, or omissions may be made to the vacuum system that includes the vacuum generation system 300 of FIG. 3 without departing from the scope of the present disclosure. For example, in some embodiments, the vacuum generation system may be further coupled to a pressure accumulator. The vacuum generation system 300 may be coupled to the vacuum accumulator 306 and open air 308 to pump air from the vacuum accumulator 306 (and thereby increase vacuum pressure within the vacuum accumulator 306) and release it to open air 308. Alternatively, the vacuum generation system 300 may be coupled to the pressure accumulator and open air 308 to pump air from the open air into the pressure accumulator (and thereby increase positive pressure within the pressure accumulator). Alternatively or additionally, the vacuum system of FIG. 3 may include one or more solenoid valves coupled between the vacuum generation system 300 and each of the vacuum accumulator, the pressure accumulator, and open air to selectively pneumatically couple the vacuum generation system 300 to the vacuum accumulator (and open air) or the pressure accumulator (and open air).

As another example, the vacuum system of FIG. 3 and/or one or more of its components (such as the vacuum generation system 202) may be coupled to a control system that may include a computing system. The control system may include, be included in, or correspond to, e.g., the system control module 110 of FIG. 1 and/or other systems or devices herein. The control system may electronically control one or more components of the vacuum system of FIG. 3.

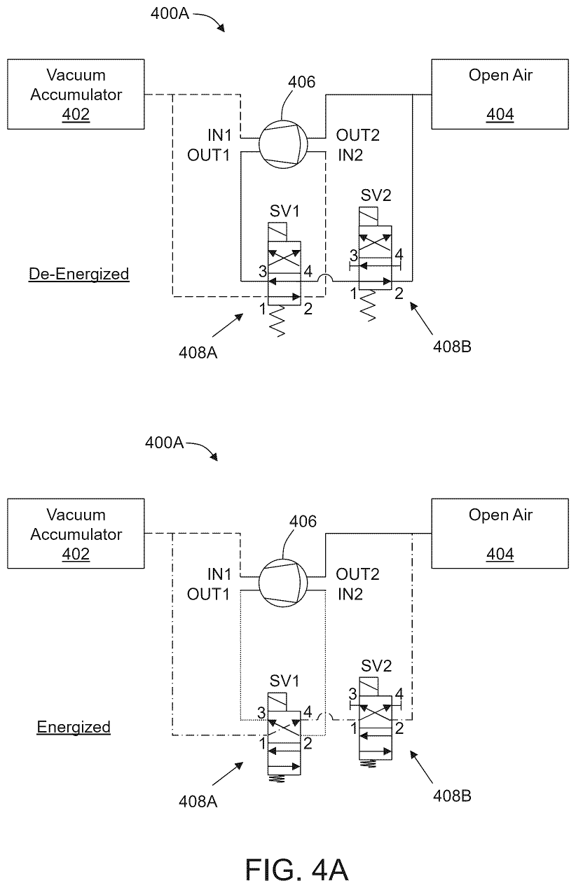

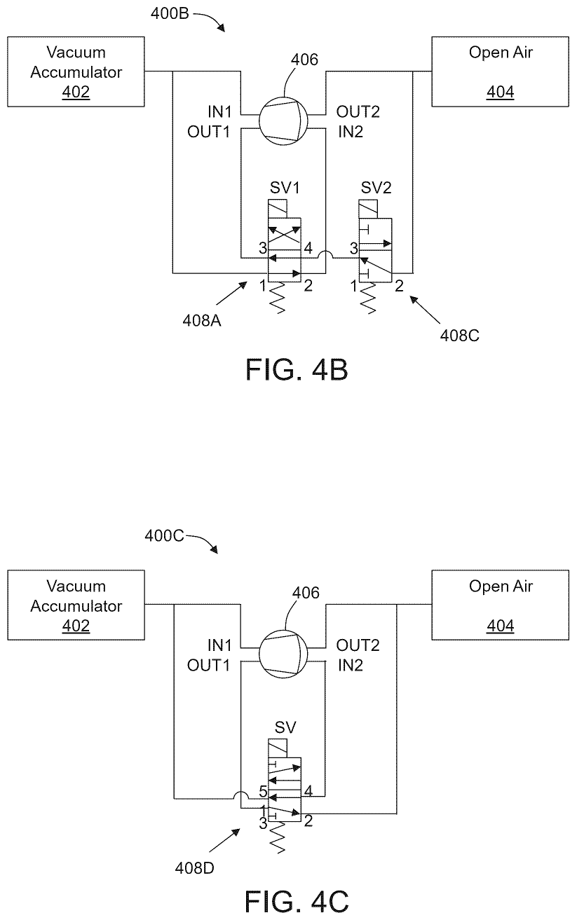

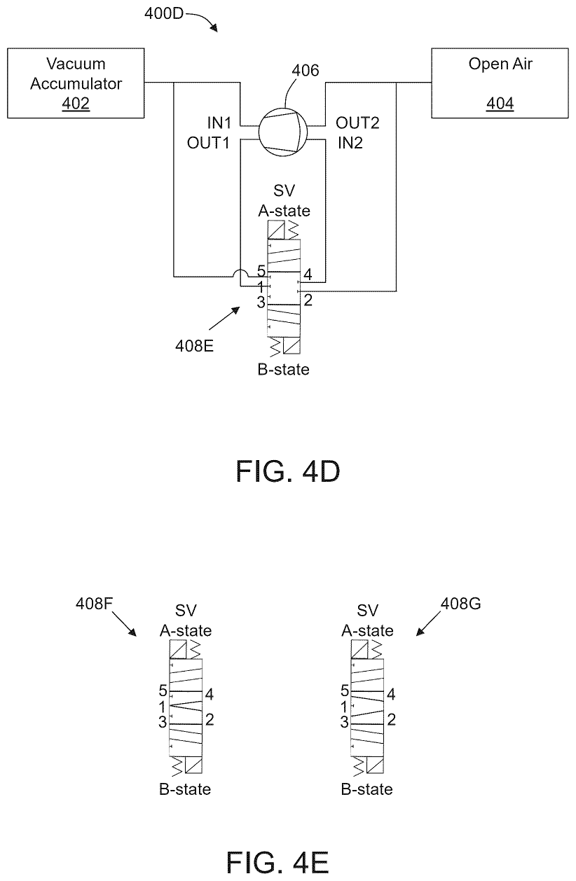

FIG. 4A is a schematic diagram of an example vacuum generation system 400A selectively operable in a parallel or series mode, arranged in accordance with at least one embodiment herein. The vacuum generation system 400A is illustrated in two states in FIG. 4A that are labeled “De-Energized” and “Energized” for reasons that will become apparent from the discussion that follows. The vacuum generation system 400A may include, be included in, or otherwise correspond to the vacuum generation system 202 of FIG. 2, the vacuum generation system 300 of FIG. 3, or other vacuum generation systems herein.

The vacuum generation system 400A may be part of a vacuum system (such as the vacuum system 200 of FIG. 2) that may further include, e.g., a vacuum accumulator 402 and open air 404 coupled to the vacuum generation system 400A. The vacuum accumulator 402 may include, be included in, or otherwise correspond to the vacuum accumulator 204 of FIG. 2 or other vacuum accumulators herein. The vacuum generation system 400A may be coupled to open air 404 through one or more intervening parts, such as a muffler or filter. Various components in FIG. 4A are connected by lines that may represent pneumatic connections.

The vacuum generation system 400A includes a multi-head diaphragm gas pump 406 (hereinafter “pump 406”) and one or more solenoid valves 408A, 408B (hereinafter generically “SV 408” or collectively “SVs 408”).

The pump 406 is illustrated as a two-head diaphragm gas pump but may more generally include multiple heads, whether the heads are part of the same multi-head diaphragm gas pump or are heads of two or more single-head diaphragm gas pumps. The pump 406 includes a first head and a second head, each including an inlet port and an outlet port. As illustrated in FIG. 4A, the first head of the pump 406 (or a first single-head diaphragm gas pump) includes a first inlet port IN1 and a first outlet port OUT1 while the second head of the pump 406 (or a second single-head diaphragm gas pump) includes a second inlet port IN2 and a second outlet port OUT2. The pump 406 may further include one or more check valves that permit one-way gas flow within each head, e.g., from IN1 to OUT1 and from IN2 to OUT2. As illustrated, IN1 of the first head of the pump 406 is pneumatically coupled to the vacuum accumulator 402 and OUT2 of the second head of the pump 406 is pneumatically coupled to open air 404.

The SVs 408 include a first SV 408A and a second SV 408B. Each of the SVs 408 is a 4-port 2-position (or 4/2) SV that includes a de-energized state and an energized state. The 4 ports of each of the SVs 408 include ports 1-4. In a first position of each of the SVs 408 associated with the de-energized state (upper half of FIG. 4A), the ports have a first set of connections therebetween. In a second position of each of the SVs 408 that is associated with the energized state (lower half of FIG. 4A), the ports have a second set of connections therebetween that are different than the first set. Thus, changing positions (e.g., by changing from the de-energized state to the energized state or vice versa) is effective to switch flows between ports. In some embodiments, each of the SVs 408 includes a spring that biases each SV 408 to the first position; energizing the SV 408 may move the SV 408 to the second position, compressing the spring (or otherwise storing energy in the spring) in the process; when the SV 408 is de-energized, the spring may decompress (or otherwise release at least some of its stored energy) to force the SV 408 back to the first position.

In FIG. 4A, the vacuum accumulator 402 is coupled to port 1 of the first SV 408A, OUT1 of the first head of the pump 406 is coupled to port 3 of the first SV 408A, IN2 of the second head of the pump 406 is coupled to port 2 of the first SV 408A, port 4 of the first SV 408A is coupled to port 1 of the second SV 408B, and port 2 of the second SV 408B is coupled to open air 404. Referring to the upper half of FIG. 4A, in the first position of each of the SVs 408 (in the de-energized state), port 1 is coupled to port 2 and port 3 is coupled to port 4. Thus, when both SVs 408 are in the de-energized state in FIG. 4A, IN1 and IN2 of the pump 406 are both pneumatically coupled to the vacuum accumulator 402 while OUT1 and OUT2 of the pump 406 are both pneumatically coupled to open air 404 such that the heads of the pump 406 are coupled in parallel between the vacuum accumulator 402 and open air 404. In the de-energized state (upper half) of FIG. 4A, the pneumatic coupling of the vacuum accumulator 402 to IN1 (directly) and IN2 (through the first SV 408A) is depicted by dashed lines while the pneumatic coupling of open air 404 to OUT2 (directly) and OUT1 (through the SVs 408) is depicted by solid lines.

Referring to the lower half of FIG. 4A, in the second position of each of the SVs 408 (in the energized state), port 1 is coupled to port 4 and port 3 is coupled to port 2. Thus, when both SVs 408 are in the energized state in FIG. 4A, IN1 of the first head of the pump 406 is pneumatically coupled to the vacuum accumulator 402, OUT1 of the first head of the pump 406 is pneumatically coupled to IN2 of the second head of the pump 406, and OUT2 of the second head of the pump 406 is pneumatically coupled to open air 404 such that the heads of the pump 406 are coupled in series between the vacuum accumulator 402 and open air 404. In the energized state (lower half) of FIG. 4A, the pneumatic coupling of the vacuum accumulator 402 to IN1 (directly) of the first head of the pump 406 is depicted by a dashed line, the pneumatic coupling of OUT1 of the first head of the pump 406 to IN2 of the second head of the pump (through the first SV 408A) is depicted by dotted lines, and the pneumatic coupling of OUT2 of the second head of the pump to open air 404 is depicted by a solid line. In the energized state, the vacuum accumulator 402 is coupled to port 4 of the second SV 408B (through ports 1 and 4 of the first SV 408A and port 1 of the second SV 408B) and open air 404 is coupled to port 3 of the second SV 408B (through port 2 of the second SV 408B) as indicated by dash-dot lines. Port 4 (and port 3 in some embodiments) of the second SV 408B may be plugged or capped as illustrated so that the vacuum accumulator 402 does not lose vacuum pressure when pneumatically coupled to port 4 of the second SV 408B in the energized state.

Modifications, additions, or omissions may be made to the vacuum system that includes the vacuum generation system 400A of FIG. 4A without departing from the scope of the present disclosure. For example, in some embodiments, the vacuum system further includes a pressure accumulator and one or more other SVs to selectively pneumatically couple the vacuum generation system 400A to (1) the vacuum accumulator 402 and open air 404 or (2) the pressure accumulator and open air 404. Alternatively or additionally, the pneumatic lines and/or the SVs 408 may be arranged to selectively pneumatically couple the heads of the pump 406 in series in the de-energized state and in parallel in the energized state.

As another example, the vacuum system of FIG. 4A and/or one or more of its components (such as the vacuum generation system 400A) may be coupled to a control system that may include a computing system. The control system may include, be included in, or correspond to, e.g., the system control module 110 of FIG. 1 and/or other systems or devices herein. The control system may electronically control one or more components of the vacuum system of FIG. 4A.

FIG. 4B is a schematic diagram of another example vacuum generation system 400B selectively operable in a parallel or series mode, arranged in accordance with at least one embodiment herein. The vacuum generation system 400B may include, be included in, or otherwise correspond to the vacuum generation system 202 of FIG. 2, the vacuum generation system 300 of FIG. 3, or other vacuum generation systems herein.

The vacuum generation system 400B may be part of a vacuum system (such as the vacuum system 200 of FIG. 2) that may further include, e.g., the vacuum accumulator 402 and open air 404 coupled to the vacuum generation system 400B. The vacuum generation system 400B may be coupled to open air 404 through one or more intervening parts, such as a muffler or filter. Various components in FIG. 4B are connected by lines that may represent pneumatic connections.

The vacuum generation system 400B includes the pump 406 and one or more SVs 408A, 408C (hereinafter generically “SV 408” or collectively “SVs 408”). The SVs 408 include the first SV 408A and a third SV 408C. While the first SV 408A is a 4/2 SV, the third SV 408C is a 3/2 SV that similarly includes a de-energized state and an energized state. The 3 ports of the third SV 408C include ports 1-3. Similar to the first SV 408A, the third SV 408C has both a first position (associated with a de-energized state) with a first set of connections between ports and a second position (associated with an energized state) with a second set of connections between ports. In the first position (associated with the de-energized state) illustrated in FIG. 4B, port 1 of the third SV 408C is internally (e.g., within the third SV 408C) plugged or capped and ports 2 and 3 of the third SV 408C are coupled together. In the second position (associated with the energized state, not illustrated in FIG. 4B), ports 1 and 2 of the third SV 408C are coupled together and port 3 of the third SV 408C is internally (e.g., within the third SV 408C) plugged or capped. Accordingly, and similar to the first SV 408A, changing positions (e.g., by changing from the de-energized state to the energized state or vice versa) of the third SV 408C is effective to switch flows between ports of the third SV 408C. In some embodiments, each of the SVs 408 of FIG. 4B includes a spring that biases each SV 408 to the first position; energizing the SV 408 may move the SV 408 to the second position, compressing the spring (or otherwise storing energy in the spring) in the process; when the SV 408 is de-energized, the spring may decompress (or otherwise release at least some of its stored energy) to force the SV 408 back to the first position.

In FIG. 4B, the vacuum accumulator 402 is coupled to port 1 of the first SV 408A, OUT1 of the first head of the pump 406 is coupled to port 3 of the first SV 408A, IN2 of the second head of the pump 406 is coupled to port 2 of the first SV 408A, port 4 of the first SV 408A is coupled to port 3 of the third SV 408C, and port 2 of the third SV 408C is coupled to open air 404. As illustrated in FIG. 4B, when both SVs 408 are in the de-energized state, IN1 and IN2 of the pump 406 are both pneumatically coupled to the vacuum accumulator 402 while OUT1 and OUT2 of the pump 406 are both pneumatically coupled to open air 404 such that the heads of the pump 406 are coupled in parallel between the vacuum accumulator 402 and open air 404.

Although not illustrated in FIG. 4B, in the second position of each of the SVs 408 (in the energized state), when both SVs 408 are in the energized state, IN1 of the first head of the pump 406 is pneumatically coupled to the vacuum accumulator 402, OUT1 of the first head of the pump 406 is pneumatically coupled to IN2 of the second head of the pump 406, and OUT2 of the second head of the pump 406 is pneumatically coupled to open air 404 such that the heads of the pump 406 are coupled in series between the vacuum accumulator 402 and open air 404. In the energized state (not illustrated in FIG. 4B), the vacuum accumulator 402 is coupled (through ports 1 and 4 of the first SV 408A) to port 3 of the third SV 408C (which is internally plugged or capped in the second position) and open air 404 is coupled to port 1 of the third SV 408C (through port 2 of the third SV 408C). In effect, the energized state of the third SVC 408C prevents flow through the third solenoid valve such that the sole path from the pump 406 to the open air 404 is via OUT2 and the sole path for OUT1 is to IN2. Port 4 (and port 3 in some embodiments) of the second SV 408C may be plugged or capped as illustrated so that the vacuum accumulator 402 does not lose vacuum pressure when pneumatically coupled to port 4 of the second SV 408C in the energized state.

Modifications, additions, or omissions may be made to the vacuum system that includes the vacuum generation system 400B of FIG. 4B without departing from the scope of the present disclosure. For example, in some embodiments, the vacuum system further includes a pressure accumulator and one or more other SVs to selectively pneumatically couple the vacuum generation system 400B to (1) the vacuum accumulator 402 and open air 404 or (2) the pressure accumulator and open air 404. Alternatively or additionally, the pneumatic lines and/or the SVs 408 may be arranged to selectively pneumatically couple the heads of the pump 406 in series in the de-energized state and in parallel in the energized state.

As another example, the vacuum system of FIG. 4B and/or one or more of its components (such as the vacuum generation system 400B) may be coupled to a control system that may include a computing system. The control system may include, be included in, or correspond to, e.g., the system control module 110 of FIG. 1 and/or other systems or devices herein. The control system may electronically control one or more components of the vacuum system of FIG. 4B.

FIG. 4C is a schematic diagram of another example vacuum generation system 400C selectively operable in a parallel or series mode, arranged in accordance with at least one embodiment herein. The vacuum generation system 400C may include, be included in, or otherwise correspond to the vacuum generation system 202 of FIG. 2, the vacuum generation system 300 of FIG. 3, or other vacuum generation systems herein.

The vacuum generation system 400C may be part of a vacuum system (such as the vacuum system 200 of FIG. 2) that may further include, e.g., the vacuum accumulator 402 and open air 404 coupled to the vacuum generation system 400C. The vacuum generation system 400C may be coupled to open air 404 through one or more intervening parts, such as a muffler or filter. Various components in FIG. 4C are connected by lines that may represent pneumatic connections.

The vacuum generation system 400C includes the pump 406 and a fourth SV 408D. The fourth SV 408D includes a 5/2 SV in the example of FIG. 4C. The 5 ports of the fourth SV 408D include ports 1-5. Similar to other SVs herein, the fourth SV 408D has both a first position (associated with a de-energized state) with a first set of connections between ports and a second position (associated with an energized state) with a second set of connections between ports. In the first position (associated with the de-energized state) illustrated in FIG. 4C, ports 1 and 2 of the fourth SV 408D are coupled together, port 3 of the fourth SV 408D is internally (e.g., within the fourth SV 408D) plugged or capped, and ports 4 and 5 of the fourth SV 408D are coupled together. In the second position (associated with the energized state, not illustrated in FIG. 4C), ports 1 and 4 of the fourth SV 408D are coupled together, ports 2 and 3 of the fourth SV 408D are coupled together, and port 5 of the fourth SV 408D is internally (e.g., within the fourth SV 408D) plugged or capped. Accordingly, and similar to other SV herein, changing positions (e.g., by changing from the de-energized state to the energized state or vice versa) of the fourth SV 408D is effective to switch flows between ports of the fourth SV 408D. In some embodiments, the fourth SV 408D of FIG. 4C includes a spring that biases the fourth SV 408D to the first position; energizing the fourth SV 408D may move the fourth SV 408D to the second position, compressing the spring (or otherwise storing energy in the spring) in the process; when the fourth SV 408D is de-energized, the spring may decompress (or otherwise release at least some of its stored energy) to force the fourth SV 408D back to the first position.

In FIG. 4C, the vacuum accumulator 402 is coupled to port 5 of the fourth SV 408D, OUT1 of the first head of the pump 406 is coupled to port 1 of the fourth SV 408D, IN2 of the second head of the pump 406 is coupled to port 4 of the fourth SV 408A, and port 2 of the fourth SV 408D is coupled to open air 404. As illustrated in FIG. 4C, when the fourth SV 408D is in the de-energized state, IN1 and IN2 of the pump 406 are both pneumatically coupled to the vacuum accumulator 402 while OUT1 and OUT2 of the pump 406 are both pneumatically coupled to open air 404 such that the heads of the pump 406 are coupled in parallel between the vacuum accumulator 402 and open air 404.

Although not illustrated in FIG. 4C, in the second position of the fourth SV 408D, when the fourth SV 408D is in the energized state, IN1 of the first head of the pump 406 is pneumatically coupled to the vacuum accumulator 402, OUT1 of the first head of the pump 406 is pneumatically coupled to IN2 of the second head of the pump 406, and OUT2 of the second head of the pump 406 is pneumatically coupled to open air 404 such that the heads of the pump 406 are coupled in series between the vacuum accumulator 402 and open air 404. In the energized state (not illustrated in FIG. 4C), the vacuum accumulator 402 remains coupled to port 5 of the fourth SV 408D which port 5 is internally plugged or blocked in the second position associated with the energized state so that the vacuum accumulator 402 does not lose vacuum pressure. Further in the energized state, OUT1 of the first head of the pump 406 is coupled (through ports 1 and 4 of the fourth SV 408D) to IN2 of the second head of the pump 406. Further still in the energized state and the associated second position, open air 404 is coupled to port 3 of the fourth SV 408D (through port 2 of the fourth SV 408D).

Modifications, additions, or omissions may be made to the vacuum system that includes the vacuum generation system 400C of FIG. 4C without departing from the scope of the present disclosure. For example, in some embodiments, the vacuum system further includes a pressure accumulator and one or more other SVs to selectively pneumatically couple the vacuum generation system 400C to (1) the vacuum accumulator 402 and open air 404 or (2) the pressure accumulator and open air 404. Alternatively or additionally, the pneumatic lines and/or the fourth SV 408D may be arranged to selectively pneumatically couple the heads of the pump 406 in series in the de-energized state and in parallel in the energized state.

As another example, the vacuum system of FIG. 4C and/or one or more of its components (such as the vacuum generation system 400C) may be coupled to a control system that may include a computing system. The control system may include, be included in, or correspond to, e.g., the system control module 110 of FIG. 1 and/or other systems or devices herein. The control system may electronically control one or more components of the vacuum system of FIG. 4B.

FIG. 4D is a schematic diagram of another example vacuum generation system 400D selectively operable in a parallel or series mode, arranged in accordance with at least one embodiment herein. The vacuum generation system 400D may include, be included in, or otherwise correspond to the vacuum generation system 202 of FIG. 2, the vacuum generation system 300 of FIG. 3, or other vacuum generation systems herein.

The vacuum generation system 400D may be part of a vacuum system (such as the vacuum system 200 of FIG. 2) that may further include, e.g., the vacuum accumulator 402 and open air 404 coupled to the vacuum generation system 400D. The vacuum generation system 400D may be coupled to open air 404 through one or more intervening parts, such as a muffler or filter. Various components in FIG. 4D are connected by lines that may represent pneumatic connections.

The vacuum generation system 400D includes the pump 406 and a fifth SV 408E. The fifth SV 408E in the example of FIG. 4D includes a 5-port 3-position (or 5/3) SV with a de-energized or neutral state and two energized states. The two energized states may be referred to as an A state and a B state. The fifth SV 408E may include two solenoid valves and two spring returns to control between the de-energized state, the A state, and the B state. The 5 ports of the fifth SV 408E include ports 1-5. Analogous to other SVs herein, the fifth SV 408E has a first position (associated with a de-energized state) with a first set of connections between ports, a second position (associated with an energized A-state) with a second set of connections between ports, and a third position (associated with an energized B-state) with a third set of connections between ports. In the first position (associated with the de-energized state) illustrated in FIG. 4D, ports 1-5 are each internally plugged or capped, i.e., there are no connections across the fifth SV 408E. In the second position (associated with the energized A-state), ports 1 and 4 of the fifth SV 408E are coupled together, ports 3 and 2 of the fifth SV 408E are coupled together, and port 5 of the fifth SV 408E is capped. In the third position (associated with the energized B-state), ports 1 and 2 of the fifth SV 408E are coupled together, ports 5 and 4 of the fifth SV 408E are coupled together, and port 3 of the fifth SV 408E is capped. Accordingly, and similar to other SVs herein, changing positions (e.g., by changing between the de-energized state, the energized A-state, and the energized B-state) of the fifth SV 408E is effective to switch flows between ports of the fifth SV 408E. In some embodiments, the fifth SV 408E of FIG. 4D includes two springs that bias the fifth SV 408E to the first position (i.e., the de-energized position); energizing the fifth SV 408E (to a given one of two different levels) may move the fifth SV 408E to the second position or the third position, compressing one of the springs and stretching the other spring (or otherwise storing energy in the springs) in the process; when the fifth SV 408E is de-energized, the compressed spring may decompress and the stretched spring may contract (or otherwise release at least some of their stored energy) to force the fifth SV 408E back to the first position.

In FIG. 4D, the vacuum accumulator 402 is coupled to port 5 of the fifth SV 408E, OUT1 of the first head of the pump 406 is coupled to port 1 of the fifth SV 408E, IN2 of the second head of the pump 406 is coupled to port 4 of the fourth SV 408A, and port 2 of the fifth SV 408E is coupled to open air 404. As illustrated in FIG. 4D, when the fifth SV 408E is in the de-energized state, the heads of the pump 406 are neither coupled in parallel nor in series.

Although not illustrated in FIG. 4D, in the second position of the fifth SV 408E, when the fifth SV 408E is in the energized A-state, IN1 of the first head of the pump 406 is pneumatically coupled to the vacuum accumulator 402, OUT1 of the first head of the pump 406 is pneumatically coupled to IN2 of the second head of the pump 406, and OUT2 of the second head of the pump 406 is pneumatically coupled to open air 404 such that the heads of the pump 406 are coupled in series between the vacuum accumulator 402 and open air 404. In the energized A-state (not illustrated in FIG. 4D), the vacuum accumulator 402 remains coupled to port 5 of the fifth SV 408E which port 5 is internally plugged or blocked in the second position associated with the energized A-state so that the vacuum accumulator 402 does not lose vacuum pressure. Further in the energized A-state, OUT1 of the first head of the pump 406 is coupled (through ports 1 and 4 of the fifth SV 408E) to IN2 of the second head of the pump 406. Further still in the energized A-state and the associated second position, open air 404 is coupled to port 3 of the fifth SV 408E (through port 2 of the fifth SV 408E).

Although not illustrated in FIG. 4D, in the third position of the fifth SV 408E, when the fifth SV 408E is in the energized B-state, IN1 and IN2 of the pump 406 are both pneumatically coupled to the vacuum accumulator 402 while OUT1 and OUT2 of the pump 406 are both pneumatically coupled to open air 404 such that the heads of the pump 406 are coupled in parallel between the vacuum accumulator 402 and open air 404. In particular, in the energized B-state (not illustrated in FIG. 4D), the vacuum accumulator 402 remains coupled to port 5 of the fifth SV 408E which port 5 is coupled to port 4 in the third position associated with the energized B-state and thereby to IN2 of the second head of the pump 406. Further in the energized B-state, OUT1 of the first head of the pump 406 is coupled (through ports 1 and 2 of the fifth SV 408E in the third position) to open air 404.

Modifications, additions, or omissions may be made to the vacuum system that includes the vacuum generation system 400D of FIG. 4D without departing from the scope of the present disclosure. For example, in some embodiments, the vacuum system further includes a pressure accumulator and one or more other SVs to selectively pneumatically couple the vacuum generation system 400D to (1) the vacuum accumulator 402 and open air 404 or (2) the pressure accumulator and open air 404. Alternatively or additionally, the pneumatic lines and/or the fifth SV 408E may be arranged to selectively pneumatically couple the heads of the pump 406 in series in the energized B-state and in parallel in the energized A-state.

As another example, the vacuum system of FIG. 4D and/or one or more of its components (such as the vacuum generation system 400D) may be coupled to a control system that may include a computing system. The control system may include, be included in, or correspond to, e.g., the system control module 110 of FIG. 1 and/or other systems or devices herein. The control system may electronically control one or more components of the vacuum system of FIG. 4B.

As yet another example, the 5/3 SV implemented as the fifth SV 408E in FIG. 4D may be any suitable type of 5/3 SV. The 5/3 SV depicted in FIG. 4D as the fifth SV 408E is one type of 5/3 SV. FIG. 4E is a schematic diagram of two other types of 5/3 SVs, respectively referred to as a sixth SV 408F and a seventh SV 408G, that may be implemented in any of the vacuum systems herein, for example, in place of the fourth SV 408D and/or the fifth SV 408E in FIG. 4C or 4D, respectively. The second and third positions of the SVs 408F, 408G respectively associated with the energized A-state and the energized B-state are identical to the second and third positions of the fifth SV 408E. In particular, in the second position associated with the energized A-state of all three SVs 408E-408G, port 5 is internally capped, port 1 is connected to port 4, and port 3 is connected to port 2. Further, in the third position associated with the energized B-state of all three SVs 408E-408G, port 3 is internally capped, port 5 is connected to port 4, and port 1 is connected to port 2. In the first position associated with the de-energized state of the sixth SV 408F of FIG. 4E, port 1 is coupled to both of ports 2 and 4. In the de-energized state of the sixth SV 408F, and assuming the pump 406 is turned off, air does not flow through the vacuum generation system 400D since the vacuum accumulator 402 is capped at port 5 and IN1 and the pump 406 is turned off. In the first position associated with the de-energized state of the seventh SV 408G of FIG. 4E, port 5 is coupled to port 4 and port 3 is coupled to port 2. In the de-energized state of the seventh SV 408G, and assuming the pump 406 is turned off, air does not flow through the vacuum generation system 400D since the vacuum accumulator 402 is capped at IN2 (via ports 5 and 4) and IN1 and the pump 406 is turned off.

FIG. 5A is a schematic diagram of an example vacuum generation system 500A selectively operable to generate vacuum pressure or positive pressure, arranged in accordance with at least one embodiment herein. The vacuum generation system 500A may include, be included in, or otherwise correspond to the vacuum generation system 202 of FIG. 2, the vacuum generation system 300 of FIG. 3, or other vacuum generation systems herein, and may include the pump 406. As illustrated in FIG. 5A, the first and second heads of the pump 406 are coupled in parallel.

The vacuum generation system 500A may be part of a vacuum system (such as the vacuum system 200 of FIG. 2) that may further include, e.g., the vacuum accumulator 402, open air 404, a pressure accumulator 510, and one or more SVs 512A, 512B (hereinafter collectively “SVs 512” or generically “SV 512”). The pressure accumulator 510 may include, be included in, or otherwise correspond to other pressure accumulators herein. The vacuum generation system 500A may be coupled to open air 404 through one or more intervening parts, such as a muffler or filter. Various components in FIG. 5A are connected by lines that may represent pneumatic connections.

The SVs 512 are generally configured to selectively pneumatically couple the vacuum generation system 500A to (1) the vacuum accumulator 402 and open air 404, or (2) the pressure accumulator 510 and open air 404. For example, in a de-energized state, the SVs 512 may pneumatically couple IN1 and IN2 of the pump 406 to open air 404 and OUT1 and OUT2 of the pump 406 to the pressure accumulator 510 such that the pump 406 may pump air from open air 404 into the pressure accumulator 510 to increase positive pressure in the pressure accumulator 510. On the other hand, in an energized state, the SVs 512 may pneumatically couple IN1 and IN2 of the pump 406 to the vacuum accumulator 402 and OUT1 and OUT2 of the pump 406 to open air 404 such that the pump 406 may pump air out of the vacuum accumulator 402 into open air 404 to increase vacuum pressure in the vacuum accumulator 402.

The SVs 512 include a first SV 512A and a second SV 512B. Each of the SVs 512 is a 4/2 SV that includes a de-energized state and an energized state. The 4 ports of each of the SVs 512 include ports 1-4. In a first position of each of the SVs 512 associated with the de-energized state (illustrated in FIG. 5A), the ports have a first set of connections therebetween. In a second position of each of the SVs 512 that is associated with the energized state (not illustrated in FIG. 5A), the ports have a second set of connections therebetween that are different than the first set. Thus, changing positions (e.g., by changing from the de-energized state to the energized state or vice versa) is effective to switch flows between ports. In some embodiments, each of the SVs 512 includes a spring that biases each SV 512 to the first position; energizing the SV 512 may move the SV 512 to the second position, compressing the spring (or otherwise storing energy in the spring) in the process; when the SV 512 is de-energized, the spring may decompress (or otherwise release at least some of its stored energy) to force the SV 512 back to the first position.

In the de-energized state of the SVs 512 illustrated in FIG. 5A, IN1 and IN2 are pneumatically coupled through ports 3 and 4 of the first SV 512A to open air 404 and OUT1 and OUT2 are pneumatically coupled through ports 1 and 2 of the first SV 512A and through ports 1 and 2 of the second SV 512B to the pressure accumulator 510. Stated another way, the first and second heads of the pump 406 are pneumatically coupled in parallel between open air 404 and the pressure accumulator 510. Further, the vacuum accumulator 402 is pneumatically coupled through port 4 of the second SV 512B to port 3 of the second SV 512B and port 3 of the second SV 512B is plugged or capped in the de-energized state to prevent loss of vacuum pressure from the vacuum accumulator 402 through the second SV 512B.

In the energized state (not illustrated in FIG. 5A) of the SVs 512, IN1 and IN2 are pneumatically coupled through ports 3 and 2 of the first SV 512A and through ports 1 and 4 of the second SV 512B to the vacuum accumulator 402 and OUT1 and OUT2 are pneumatically coupled through ports 1 and 4 of the first SV 512A to open air 404. Stated another way, the first and second heads of the pump 406 are pneumatically coupled in parallel between open air 404 and the vacuum accumulator 402. Further, the pressure accumulator 510 is pneumatically coupled through port 2 of the second SV 512B to port 3 of the second SV 512B and port 3 of the second SV 512B is plugged or capped in the energized state to prevent loss of positive pressure from the pressure accumulator 510 through the second SV 512B.

Modifications, additions, or omissions may be made to the vacuum system that includes the vacuum generation system 500A of FIG. 5A without departing from the scope of the present disclosure. For example, in some embodiments, the vacuum system further includes one or more other SVs to selectively pneumatically couple the heads of the pump 406 in parallel or series. Alternatively or additionally, the pneumatic lines and/or the SVs 512 may be arranged to selectively pneumatically couple the vacuum generation system 500A to the vacuum accumulator 402 and open air 404 in the de-energized state and to the pressure accumulator 510 and open air 404 in the energized state.

As another example, the vacuum system of FIG. 5A and/or one or more of its components (such as the vacuum generation system 500A) may be coupled to a control system that may include a computing system. The control system may include, be included in, or correspond to, e.g., the system control module 110 of FIG. 1 and/or other systems or devices herein. The control system may electronically control one or more components of the vacuum system of FIG. 5A.

FIG. 5B is a schematic diagram of another example vacuum generation system 500B selectively operable to generate vacuum pressure or positive pressure, arranged in accordance with at least one embodiment herein. The vacuum generation system 500B may include, be included in, or otherwise correspond to the vacuum generation system 202 of FIG. 2, the vacuum generation system 300 of FIG. 3, or other vacuum generation systems herein.

The vacuum generation system 500B may be part of a vacuum system (such as the vacuum system 200 of FIG. 2) that may further include, e.g., the vacuum accumulator 402, open air 404, the pressure accumulator 510, and one or more SVs 512A, 512C (hereinafter collectively “SVs 512” or generically “SV 512”). The vacuum generation system 500B may be coupled to open air 404 through one or more intervening parts, such as a muffler or filter. Various components in FIG. 5B are connected by lines that may represent pneumatic connections.

The SVs 512 of FIG. 5B are generally configured to selectively pneumatically couple the vacuum generation system 500B to (1) the vacuum accumulator 402 and open air 404, or (2) the pressure accumulator 510 and open air 404. For example, and as illustrated in FIG. 5B, in a de-energized state, the SVs 512 may pneumatically couple IN1 and IN2 of the pump 406 to open air 404 and OUT1 and OUT2 of the pump 406 to the pressure accumulator 510 such that the pump 406 may pump air from open air 404 into the pressure accumulator 510 to increase positive pressure in the pressure accumulator 510. On the other hand, in an energized state, the SVs 512 may pneumatically couple IN1 and IN2 of the pump 406 to the vacuum accumulator 402 and OUT1 and OUT2 of the pump 406 to open air 404 such that the pump 406 may pump air out of the vacuum accumulator 402 into open air 404 to increase vacuum pressure in the vacuum accumulator 402.

The SVs 512 include the first SV 512A and a third SV 512C. While the first SV 512A is a 4/2 SV, the third SV 512C is instead a 3/2 SV, although both SVs 512A, 512C include a de-energized state and an energized state. The 3 ports of the third SV 512C include ports 1-3. Similar to the first SV 512A, in a first position of the third SV 512C associated with the de-energized state (illustrated in FIG. 5B), the 3 ports have a first set of connections therebetween. In a second position of the third SV 512C that is associated with the energized state (not illustrated in FIG. 5B), the 3 ports have a second set of connections therebetween that are different than the first set. In the first position (associated with the de-energized state) illustrated in FIG. 5B, port 1 of the third SV 512C is internally plugged or capped and ports 2 and 3 of the third SV 512C are coupled together. In the second position (associated with the energized state, not illustrated in FIG. 5B), ports 1 and 2 of the third SV 512C are coupled together and port 3 of the third SV 512C is internally plugged or capped. Accordingly, and similar to the first SV 512A, changing positions (e.g., by changing from the de-energized state to the energized state or vice versa) of the third SV 512C is effective to switch flows between ports of the third SV 512C. In some embodiments, the third SV 512C includes a spring that biases the third SV 512C to the first position; energizing the third SV 512C may move the third SV 512C to the second position, compressing the spring (or otherwise storing energy in the spring) in the process; when the third SV 512C is de-energized, the spring may decompress (or otherwise release at least some of its stored energy) to force the third SV 512C back to the first position.

In the de-energized state of the SVs 512 illustrated in FIG. 5B, IN1 and IN2 are pneumatically coupled through ports 3 and 4 of the first SV 512A to open air 404 and OUT1 and OUT2 are pneumatically coupled through ports 1 and 2 of the first SV 512A and through ports 2 and 3 of the third SV 512C to the pressure accumulator 510. Stated another way, the first and second heads of the pump 406 are pneumatically coupled in parallel between open air 404 and the pressure accumulator 510. Further, the vacuum accumulator 402 is pneumatically coupled to port 1 of the third SV 512C which may be internally plugged or capped in the de-energized state to prevent loss of vacuum pressure from the vacuum accumulator 402 through the third SV 512C.

In the energized state (not illustrated) of the SVs 512 of FIG. 5B, IN1 and IN2 are pneumatically coupled through ports 3 and 2 of the first SV 512A and through ports 2 and 1 of the third SV 512C to the vacuum accumulator 402 and OUT1 and OUT2 are pneumatically coupled through ports 1 and 4 of the first SV 512A to open air 404. Stated another way, the first and second heads of the pump 406 are pneumatically coupled in parallel between open air 404 and the vacuum accumulator 402. Further, the pressure accumulator 510 is pneumatically coupled to port 1 of the third SV 512C which may be internally plugged or capped in the energized state to prevent loss of positive pressure from the pressure accumulator 510 through the third SV 512C.

Modifications, additions, or omissions may be made to the vacuum system that includes the vacuum generation system 500B of FIG. 5B without departing from the scope of the present disclosure. For example, in some embodiments, the vacuum system further includes one or more other SVs to selectively pneumatically couple the heads of the pump 406 in parallel or series. Alternatively or additionally, the pneumatic lines and/or the SVs 512 may be arranged to selectively pneumatically couple the vacuum generation system 500B to the vacuum accumulator 402 and open air 404 in the de-energized state and to the pressure accumulator 510 and open air 404 in the energized state.

As another example, the vacuum system of FIG. 5B and/or one or more of its components (such as the vacuum generation system 500B) may be coupled to a control system that may include a computing system. The control system may include, be included in, or correspond to, e.g., the system control module 110 of FIG. 1 and/or other systems or devices herein. The control system may electronically control one or more components of the vacuum system of FIG. 5B.

FIG. 5C is a schematic diagram of another example vacuum generation system 500C selectively operable to generate vacuum pressure or positive pressure, arranged in accordance with at least one embodiment herein. The vacuum generation system 500C may include, be included in, or otherwise correspond to the vacuum generation system 202 of FIG. 2, the vacuum generation system 300 of FIG. 3, or other vacuum generation systems herein.

The vacuum generation system 500C may be part of a vacuum system (such as the vacuum system 200 of FIG. 2) that may further include, e.g., the vacuum accumulator 402, open air 404, the pressure accumulator 510, and a fourth SV 512D. The vacuum generation system 500C may be coupled to open air 404 through one or more intervening parts, such as a muffler or filter. Various components in FIG. 5C are connected by lines that may represent pneumatic connections.

The fourth SV 512D includes a 5/2 SV in the example of FIG. 5C. The 5 ports of the fourth SV 512D include ports 1-5. Similar to other SVs herein, the fourth SV 512D has both a first position (associated with a de-energized state) with a first set of connections between ports and a second position (associated with an energized state) with a second set of connections between ports. In the first position (associated with the de-energized state) illustrated in FIG. 5C, ports 1 and 2 of the fourth SV 512D are coupled together, port 3 of the fourth SV 512D is internally plugged or capped, and ports 4 and 5 of the fourth SV 512D are coupled together. In the second position (associated with the energized state, not illustrated in FIG. 5C), ports 1 and 4 of the fourth SV 512D are coupled together, ports 2 and 3 of the fourth SV 512D are coupled together, and port 5 of the fourth SV 512D is internally plugged or capped. Accordingly, and similar to other SVs herein, changing positions (e.g., by changing from the de-energized state to the energized state or vice versa) of the fourth SV 512D is effective to switch flows between ports of the fourth SV 512D. In some embodiments, the fourth SV 512D of FIG. 5C includes a spring that biases the fourth SV 512D to the first position; energizing the fourth SV 512D may move the fourth SV 512D to the second position, compressing the spring (or otherwise storing energy in the spring) in the process; when the fourth SV 512D is de-energized, the spring may decompress (or otherwise release at least some of its stored energy) to force the fourth SV 512D back to the first position.

The fourth SV 512D of FIG. 5C is generally configured to selectively pneumatically couple the vacuum generation system 500C to (1) the vacuum accumulator 402 and open air 404, or (2) the pressure accumulator 510 and open air 404. For example, and as illustrated in FIG. 5C, in a de-energized state, the fourth SV 512D may pneumatically couple IN1 and IN2 of the pump 406 through ports 1 and 2 of the fourth SV 512D to open air 404 and OUT1 and OUT2 of the pump 406 through ports 4 and 5 of the fourth SV 512D to the pressure accumulator 510 such that the pump 406 may pump air from open air 404 into the pressure accumulator 510 to increase positive pressure in the pressure accumulator 510. Further, the vacuum accumulator 402 is pneumatically coupled to port 3 of the fourth SV 512D which may be internally plugged or capped in the de-energized state to prevent loss of vacuum pressure from the vacuum accumulator 402 through the fourth SV 512D.

On the other hand, in an energized state, the fourth SV 512D may pneumatically couple IN1 and IN2 of the pump 406 through ports 2 and 3 of the fourth SV 512D to the vacuum accumulator 402 and OUT1 and OUT2 of the pump 406 through ports 1 and 4 of the fourth SV 512D to open air 404 such that the pump 406 may pump air out of the vacuum accumulator 402 into open air 404 to increase vacuum pressure in the vacuum accumulator 402. Further, the pressure accumulator 510 is pneumatically coupled to port 5 of the fourth SV 512D which may be internally plugged or capped in the energized state to prevent loss of positive pressure from the pressure accumulator 510 through the fourth SV 512D.

Modifications, additions, or omissions may be made to the vacuum system that includes the vacuum generation system 500C of FIG. 5C without departing from the scope of the present disclosure. For example, in some embodiments, the vacuum system further includes one or more other SVs to selectively pneumatically couple the heads of the pump 406 in parallel or series. Alternatively or additionally, the pneumatic lines and/or the SVs 512 may be arranged to selectively pneumatically couple the vacuum generation system 500C to the vacuum accumulator 402 and open air 404 in the de-energized state and to the pressure accumulator 510 and open air 404 in the energized state.

As another example, the vacuum system of FIG. 5C and/or one or more of its components (such as the vacuum generation system 500C) may be coupled to a control system that may include a computing system. The control system may include, be included in, or correspond to, e.g., the system control module 110 of FIG. 1 and/or other systems or devices herein. The control system may electronically control one or more components of the vacuum system of FIG. 5C.

FIG. 5D is a schematic diagram of another example vacuum generation system 500D selectively operable to generate vacuum pressure or positive pressure, arranged in accordance with at least one embodiment herein. The vacuum generation system 500D may include, be included in, or otherwise correspond to the vacuum generation system 202 of FIG. 2, the vacuum generation system 300 of FIG. 3, or other vacuum generation systems herein.

The vacuum generation system 500D may be part of a vacuum system (such as the vacuum system 200 of FIG. 2) that may further include, e.g., the vacuum accumulator 402, open air 404, the pressure accumulator 510, and a fifth SV 512E. The vacuum generation system 500D may be coupled to open air 404 through one or more intervening parts, such as a muffler or filter. Various components in FIG. 5D are connected by lines that may represent pneumatic connections.

The fifth SV 512E includes a 5/3 SV in the example of FIG. 5D. The 5 ports of the fifth SV 512E include ports 1-5. Similar to other 5/3 SVs herein, the fifth SV 512E has a first position (associated with a de-energized state) with a first set of connections between ports, a second position (associated with an energized A-state) with a second set of connections between ports, and a third position (associated with an energized B-state) with a third set of connections between ports. In the first position (associated with the de-energized state) illustrated in FIG. 5D, none of the ports of the fifth SV 512E are coupled together. In the second position (associated with the energized A-state, not illustrated in FIG. 5D), ports 1 and 4 of the fifth SV 512E are coupled together, ports 2 and 3 of the fifth SV 512E are coupled together, and port 5 of the fifth SV 512E is internally plugged or capped. In the third position (associated with the energized B-state, not illustrated in FIG. 5D), ports 1 and 2 of the fifth SV 512E are coupled together, port 3 of the fifth SV 512E is internally plugged or capped, and ports 4 and 5 of the fifth SV 512E are coupled together. Accordingly, and similar to other SVs herein, changing positions (e.g., by changing between the de-energized state, the energized A-state, and the energized B-state) of the fifth SV 512E is effective to switch flows between ports of the fifth SV 512E. In some embodiments, the fifth SV 512E of FIG. 5D includes two springs that bias the fifth SV 512E to the first position; energizing the fifth SV 512E (to a given one of two different levels) may move the fifth SV 512E to the second position or the third position, compressing one of the springs and stretching the other spring (or otherwise storing energy in the springs) in the process; when the fifth SV 512E is de-energized, the compressed spring may decompress and the stretched spring may contract (or otherwise release at least some of their stored energy) to force the fifth SV 512E back to the first position.

The fifth SV 512E of FIG. 5D is generally configured to selectively pneumatically couple the vacuum generation system 500D to (1) the vacuum accumulator 402 and open air 404, or (2) the pressure accumulator 510 and open air 404. For example, in the energized B-state, the fifth SV 512E may pneumatically couple IN1 and IN2 of the pump 406 through ports 1 and 2 of the fifth SV 512E to open air 404 and OUT1 and OUT2 of the pump 406 through ports 4 and 5 of the fifth SV 512E to the pressure accumulator 510 such that the pump 406 may pump air from open air 404 into the pressure accumulator 510 to increase positive pressure in the pressure accumulator 510. Further, the vacuum accumulator 402 is pneumatically coupled to port 3 of the fifth SV 512E which may be internally plugged or capped in the energized B-state to prevent loss of vacuum pressure from the vacuum accumulator 402 through the fifth SV 512E.

On the other hand, in the energized A-state, the fifth SV 512E may pneumatically couple IN1 and IN2 of the pump 406 through ports 2 and 3 of the fifth SV 512E to the vacuum accumulator 402 and OUT1 and OUT2 of the pump 406 through ports 1 and 4 of the fifth SV 512E to open air 404 such that the pump 406 may pump air out of the vacuum accumulator 402 into open air 404 to increase vacuum pressure in the vacuum accumulator 402. Further, the pressure accumulator 510 is pneumatically coupled to port 5 of the fifth SV 512E which may be internally plugged or capped in the energized A-state to prevent loss of positive pressure from the pressure accumulator 510 through the fifth SV 512E.

Modifications, additions, or omissions may be made to the vacuum system that includes the vacuum generation system 500D of FIG. 5D without departing from the scope of the present disclosure. For example, in some embodiments, the vacuum system further includes one or more other SVs to selectively pneumatically couple the heads of the pump 406 in parallel or series. Alternatively or additionally, the pneumatic lines and/or the SVs 512 may be arranged to selectively pneumatically couple the vacuum generation system 500D to the vacuum accumulator 402 and open air 404 in the energized A-state and to the pressure accumulator 510 and open air 404 in the energized B-state.

As another example, the vacuum system of FIG. 5D and/or one or more of its components (such as the vacuum generation system 500D) may be coupled to a control system that may include a computing system. The control system may include, be included in, or correspond to, e.g., the system control module 110 of FIG. 1 and/or other systems or devices herein. The control system may electronically control one or more components of the vacuum system of FIG. 5D.

While FIGS. 4A-4D illustrate various embodiments that are selectively operable in a parallel or series mode and FIGS. 5A-5D illustrate various embodiments that are selectively operable to generate vacuum pressure or positive pressure, the embodiments may be combined in any desired manner. Some examples of such combinations, but not necessarily all such combinations, are illustrated in FIGS. 6A-6J.

FIGS. 6A-6J are schematic diagrams of various example vacuum systems 600A-600J (hereinafter generically “vacuum system 600” or collectively “vacuum systems 600”) selectively operable in a parallel or series mode and selectively operable to generate vacuum pressure or positive pressure, each arranged in accordance with at least one embodiment herein. Each vacuum system 600 may include, be included in, or otherwise correspond to the vacuum system 114 of FIG. 1, the vacuum system 200 of FIG. 2, or other vacuum systems herein.

Each vacuum system 600 may include one of the vacuum generation systems 400A, 400B, 400C, 400D of FIGS. 4A-4D. As such, each vacuum system 600 may include the pump 406 and one or more of the SVs 408A, 408B, 408C, 408D, 408E (hereinafter collectively “SVs 408” or generically “SV 408”) of FIGS. 4A-4D, or any other solenoid valve. Each vacuum system 600 may further include, e.g., the vacuum accumulator 402, open air 404, the pressure accumulator 510, and one or more of the SVs 512A, 512B, 512C, 512D, 512E (hereinafter collectively “SVs 512” or generically “SV 512”) of FIGS. 5A-5D, or any other solenoid valve. In FIGS. 6A-6J, each of the vacuum generation systems 400A, 400B, 400C, 400D may be coupled to open air 404 in the corresponding vacuum system 600 through one or more intervening parts, such as a muffler or filter. Various components in FIGS. 6A-6J are connected by lines that may represent pneumatic connections.

FIGS. 6A-6J include various arrangements of SVs 408 from FIGS. 4A-4C to selectively couple the heads of the pump 406 in parallel or series. Further, FIGS. 6A-6J include various arrangements of SVs 512 from FIGS. 5A-5D to selectively generate vacuum pressure or positive pressure. Thus, the various arrangements of the SVs 408, 512 in FIGS. 6A-6J allow the vacuum systems that include the vacuum generation systems 600 of FIGS. 6A-6J to (1) operate with the heads of the pump 406 in parallel to generate vacuum pressure, (2) operate with the heads of the pump 406 in series to generate vacuum pressure, (3) operate with the heads of the pump 406 in parallel to generate positive pressure, or (4) operate with the heads of the pump 406 in series to generate positive pressure.

FIG. 6A combines the embodiments of FIGS. 4A and 5A. In particular, in FIG. 6A, the first and second SVs 408A, 408B of FIG. 4A selectively pneumatically couple the heads of the pump 406 in parallel or series as described with respect to FIG. 4A. Further in FIG. 6A, the first and second SVs 512A, 512B of FIG. 5A selectively pneumatically couple the vacuum generation system 400A to (1) open air 404 and the pressure accumulator 510 (to generate positive pressure) or (2) open air 404 and the vacuum accumulator 402 (to generate vacuum pressure) in the same manner as in FIG. 5A.

FIG. 6B combines the embodiments of FIGS. 4B and 5B. In particular, in FIG. 6B, the first and third SVs 408A, 408C of FIG. 4B selectively pneumatically couple the heads of the pump 406 in parallel or series as described with respect to FIG. 4B. Further in FIG. 6B, the first and third SVs 512A, 512C of FIG. 5B selectively pneumatically couple the vacuum generation system 400B to (1) open air 404 and the pressure accumulator 510 (to generate positive pressure) or (2) open air 404 and the vacuum accumulator 402 (to generate vacuum pressure) in the same manner as in FIG. 5B. In this and other embodiments, port 1 of the third SV 408C may be plugged or capped so that when the heads of the pump 406 are configured in series (both SVs 408A, 408C are energized) and the vacuum generation system 400B is coupled to the pressure accumulator 510 and open air 404 (both SVs 512A, 512C are de-energized), the pressure accumulator 510 does not lose air pressure through the third SV 408C.

FIG. 6C combines the embodiments of FIGS. 4A and 5C. In particular, in FIG. 6C, the first and second SVs 408A, 408B of FIG. 4A selectively pneumatically couple the heads of the pump 406 in parallel or series as described with respect to FIG. 4A. Further in FIG. 6C, the fourth SV 512D of FIG. 5C selectively pneumatically couples the vacuum generation system 400A to (1) open air 404 and the pressure accumulator 510 (to generate positive pressure) or (2) open air 404 and the vacuum accumulator 402 (to generate vacuum pressure) in the same manner as in FIG. 5C.

FIG. 6D combines the embodiments of FIGS. 4B and 5C. In particular, in FIG. 6D, the first and third SVs 408A, 408C of FIG. 4B selectively pneumatically couple the heads of the pump 406 in parallel or series as described with respect to FIG. 4B. Further in FIG. 6D, the fourth SV 512D of FIG. 5C selectively pneumatically couples the vacuum generation system 400B to (1) open air 404 and the pressure accumulator 510 (to generate positive pressure) or (2) open air 404 and the vacuum accumulator 402 (to generate vacuum pressure) in the same manner as in FIG. 5C. In this and other embodiments, port 1 of the third SV 408C may be plugged or capped so that when the heads of the pump 406 are configured in series (both SVs 408A, 408C are energized) and the vacuum generation system 400B is coupled to the pressure accumulator 510 and open air 404 (both SVs 512A, 512C are de-energized), the pressure accumulator 510 does not lose air pressure through the third SV 408C.

FIG. 6E combines the embodiments of FIGS. 4C and 5A. In particular, in FIG. 6E, the fourth SV 408D of FIG. 4C selectively pneumatically couples the heads of the pump 406 in parallel or series as described with respect to FIG. 4C. Further in FIG. 6E, the first and second SVs 512A, 512B of FIG. 5A selectively pneumatically couple the vacuum generation system 400C to (1) open air 404 and the pressure accumulator 510 (to generate positive pressure) or (2) open air 404 and the vacuum accumulator 402 (to generate vacuum pressure) in the same manner as in FIG. 5A.

FIG. 6F combines the embodiments of FIGS. 4C and 5B. In particular, in FIG. 6F, the fourth SV 408D of FIG. 4C selectively pneumatically couples the heads of the pump 406 in parallel or series as described with respect to FIG. 4C. Further in FIG. 6F, the first and third SVs 512A, 512C of FIG. 5B selectively pneumatically couple the vacuum generation system 400C to (1) open air 404 and the pressure accumulator 510 (to generate positive pressure) or (2) open air 404 and the vacuum accumulator 402 (to generate vacuum pressure) in the same manner as in FIG. 5B.

FIG. 6G combines the embodiments of FIGS. 4A and 5B. In particular, in FIG. 6G, the first and second SVs 408A, 408B of FIG. 4A selectively pneumatically couple the heads of the pump 406 in parallel or series as described with respect to FIG. 4A. Further in FIG. 6G, the first and third SVs 512A, 512C of FIG. 5B selectively pneumatically couple the vacuum generation system 400A to (1) open air 404 and the pressure accumulator 510 (to generate positive pressure) or (2) open air 404 and the vacuum accumulator 402 (to generate vacuum pressure) in the same manner as in FIG. 5B.

FIG. 6H combines the embodiments of FIGS. 4B and 5A. In particular, in FIG. 6H, the first and third SVs 408A, 408C of FIG. 4B selectively pneumatically couple the heads of the pump 406 in parallel or series as described with respect to FIG. 4B. Further in FIG. 6H, the first and second SVs 512A, 512B of FIG. 5A selectively pneumatically couple the vacuum generation system 400C to (1) open air 404 and the pressure accumulator 510 (to generate positive pressure) or (2) open air 404 and the vacuum accumulator 402 (to generate vacuum pressure) in the same manner as in FIG. 5A. In this and other embodiments, port 1 of the third SV 408C may be plugged or capped so that when the heads of the pump 406 are configured in series (both SVs 408A, 408C are energized) and the vacuum generation system 400B is coupled to the pressure accumulator 510 and open air 404 (both SVs 512A, 512C are de-energized), the pressure accumulator 510 does not lose air pressure through the third SV 408C.

FIG. 6I combines the embodiments of FIGS. 4C and 5C. In particular, in FIG. 6I, the fourth SV 408D of FIG. 4C selectively pneumatically couples the heads of the pump 406 in parallel or series as described with respect to FIG. 4C. Further in FIG. 6I, the fourth SV 512D of FIG. 5C selectively pneumatically couples the vacuum generation system 400C to (1) open air 404 and the pressure accumulator 510 (to generate positive pressure) or (2) open air 404 and the vacuum accumulator 402 (to generate vacuum pressure) in the same manner as in FIG. 5C.

All implementations involving a 5/2 SV, such as the fourth SV 408D and/or the fourth SV 512D in FIGS. 6C-6F, 6I, may be modified to instead implement a 5/3 SV, such as the fifth SV 408E and/or the fifth SV 512E. Without illustrating all possible modifications, FIG. 6J shows one modification of FIG. 6I in which the fourth SV 512D has been replaced by the fifth SV 512E. Thus, FIG. 6J combines the embodiments of FIGS. 4C and 5D. In particular, in FIG. 6J, the fourth SV 408D of FIG. 4C selectively pneumatically couples the heads of the pump 406 in parallel or series as described with respect to FIG. 4C. Further in FIG. 6J, the fifth SV 512E of FIG. 5D selectively pneumatically couples the vacuum generation system 400C to (1) open air 404 and the pressure accumulator 510 (to generate positive pressure) or (2) open air 404 and the vacuum accumulator 402 (to generate vacuum pressure) in the same manner as in FIG. 5D.

Modifications, additions, or omissions may be made to the vacuum systems 600 of FIGS. 6A-6J without departing from the scope of the present disclosure. For example, the vacuum systems of FIGS. 6A-6J and/or one or more of their components (such as the vacuum generation systems 400A, 400B, 400C) may be coupled to a control system that may include a computing system. The control system may include, be included in, or correspond to, e.g., the system control module 110 of FIG. 1 and/or other systems or devices herein. The control system may electronically control one or more components of the vacuum systems of FIGS. 6A-6J.

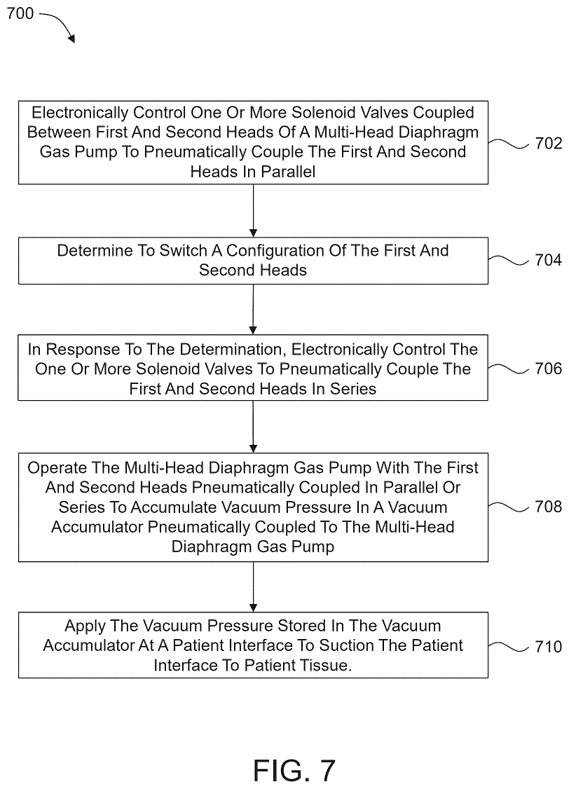

FIG. 7 is a flowchart of an example method 700 to operate a vacuum system for an ophthalmic surgical system, arranged in accordance with at least one embodiment herein. The method 700 may be performed or controlled by any suitable system, apparatus, or device. For example, the method 700 may be implemented, in whole or in part, in the ophthalmic surgical system 100 of FIG. 1, the vacuum system 114 of FIG. 1, the vacuum system 200 of FIG. 2, the vacuum generation system 300 of FIG. 3, or other system or device herein, and/or using the control module 110 of FIG. 1, the control system 312, or other control or computing system herein. Alternatively or additionally, the method 700 may be embodied in computer-readable instructions stored on a computer-readable storage medium, the computer-readable instructions executable by a processor to perform or control performance of the method 700. The method 700 may include one or more of blocks 702, 704, 706, 708, and/or 710. Although illustrated with discrete blocks, the steps and operations associated with one or more blocks of the method 700 may be divided into additional blocks, combined into fewer blocks, or eliminated, depending on the particular implementation.

At block 702, the method 700 may include electronically controlling one or more solenoid valves (e.g., one or more of SVs 408) coupled between first and second diaphragm gas pump heads (e.g., of a multi-head diaphragm gas pump such as pump 302 and/or 406, or of multiple single-head diaphragm gas pumps) to pneumatically couple the first and second heads in parallel. Block 702 may be followed by block 704.

At block 704, the method 700 may include determining to switch a configuration of the first and second heads. For example, such a determination may be based on a threshold amount of vacuum or positive pressure that has accumulated in an accumulator. As another example, such a determination may be based on a threshold pressure (e.g., barometric pressure or pressure internal to an accumulator) being detected. Block 704 may be followed by block 706.

At block 706, and in response to the determination, the method 700 may include electronically controlling the one or more solenoid valves (e.g., one or more of SVs 408) to pneumatically couple the first and second heads in series. Block 706 may be followed by block 708.

At block 708, the method 700 may include operating the multi-head diaphragm gas pump (e.g., pump 302 and/or 406) with the first and second heads pneumatically coupled in parallel or series to accumulate vacuum pressure in a vacuum accumulator (e.g., vacuum accumulator 204, 306, and/or 402) pneumatically coupled to the multi-head diaphragm gas pump. Block 708 may be followed by block 710.

At block 710, the method 700 may include applying the vacuum pressure stored in the vacuum accumulator (e.g., vacuum accumulator 204, 306, and/or 402) at a patient interface to suction the patient interface to patient tissue.

In some embodiments, operating the multi-head diaphragm gas pump with the first and second heads pneumatically coupled in parallel or series at block 708 includes operating the multi-head diaphragm gas pump with the first and second heads pneumatically coupled in parallel after electronically controlling the one or more solenoid valves coupled between the first and second heads to pneumatically couple the first and second heads in parallel at block 702 and prior to determining to switch the configuration of the first and second heads at block 704. The method 700 may further include monitoring vacuum pressure in the vacuum accumulator, while operating the multi-head diaphragm gas pump with the first and second heads pneumatically coupled in parallel, prior to determining to switch the configuration of the first and second heads. The vacuum pressure may be monitored in the vacuum accumulator using a pressure sensor, such as the pressure sensor 310 of FIG. 3. In this and other embodiments, determining to switch the configuration of the first and second heads (e.g., from parallel to series) may be based on the monitored vacuum pressure in the vacuum accumulator reaching a predetermined threshold. In some embodiments, the predetermined threshold may be determined prior to a surgical procedure, e.g., in a calibration step or process in which the predetermined threshold is determined as the pressure at which the vacuum accumulator stabilizes when the first and second heads are operated in parallel.

Alternatively or additionally, operating the multi-head diaphragm gas pump with the first and second heads pneumatically coupled in parallel or series at block 708 further includes operating the multi-head diaphragm gas pump with the first and second heads pneumatically coupled in series after electronically controlling the one or more solenoid valves to pneumatically couple the first and second heads in series at block 706. In this and other embodiments, the method 700 may further include continuing to monitor vacuum pressure in the vacuum accumulator while operating the multi-head diaphragm gas pump with the first and second heads pneumatically coupled in series. The method 700 may further include turning off the multi-head diaphragm gas pump in response to the monitored vacuum pressure in the vacuum accumulator reaching a second predetermined threshold. In some embodiments, the second predetermined threshold may be determined prior to a surgical procedure, e.g., in a calibration step or process in which the second predetermined threshold is determined as the pressure at which the vacuum accumulator stabilizes when the first and second heads are operated in series.

In other embodiments, neither the predetermined threshold nor the second predetermined threshold may be known in advance of an ophthalmic surgical procedure. Instead, the vacuum pressure may be monitored and the system may switch the first and second heads of the pump from parallel to series after the vacuum pressure stabilizes while operating in parallel and may then turn the pump off after the vacuum pressure stabilizes again while operating in series. In particular, during the ophthalmic surgical procedure, the method 700 may include monitoring vacuum pressure in the vacuum accumulator while operating the multi-head diaphragm gas pump with the first and second heads pneumatically coupled in parallel. The method 700 may include determining based on the monitored vacuum pressure in the vacuum accumulator that vacuum pressure in the vacuum accumulator has stabilized with the first and second heads pneumatically coupled in parallel. Determining to switch the configuration of the first and second heads at block 704 in this example may be based on determining that vacuum pressure in the vacuum accumulator has stabilized with the first and second heads pneumatically coupled in parallel. The method 700 may include continuing to monitor vacuum pressure in the vacuum accumulator while operating the multi-head diaphragm gas pump with the first and second heads pneumatically coupled in series. The method 700 may include determining based on the monitored vacuum pressure in the vacuum accumulator that vacuum pressure in the vacuum accumulator has stabilized with the first and second heads pneumatically coupled in series. The method 700 may include turning off the multi-head diaphragm gas pump in response to determining that vacuum pressure in the vacuum accumulator has stabilized with the first and second heads pneumatically coupled in series.

In some embodiments, the method 700 further includes electronically controlling one or more other solenoid valves (e.g., one or more of SVs 512) coupled between the vacuum generation system and each of the vacuum accumulator, a pressure accumulator, and open air to selectively pneumatically couple the vacuum generation system to the vacuum accumulator and open air or to the pressure accumulator and open air. Thus, the vacuum system may be operated to generate vacuum pressure and store it in the vacuum accumulator or to generate positive pressure and store it in the pressure accumulator. In these and other embodiments, the method 700 may include capping or blocking one or both of the pressure accumulator and/or the vacuum accumulator when a threshold amount of pressure has been reached. For example, using an SV such as SV 512E, one or both of the pressure accumulator and/or the vacuum accumulator may be capped or blocked in a de-energized state.

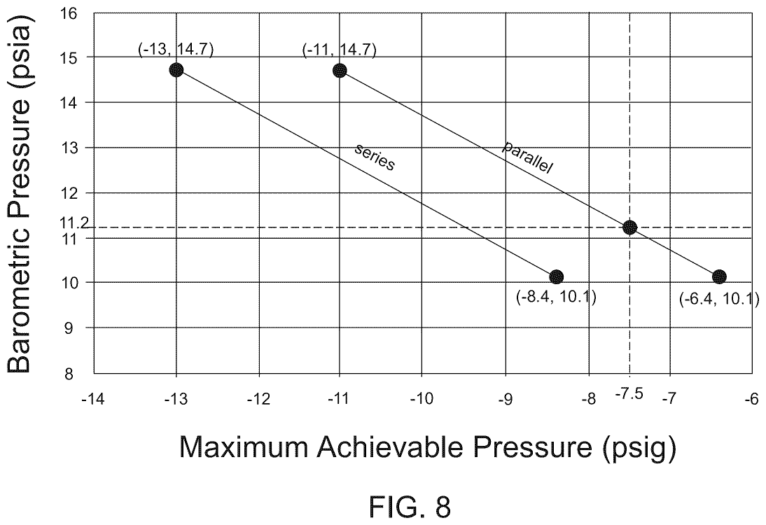

In some embodiments, the method 700 further includes receiving measured barometric pressure of a usage location of the multi-head diaphragm gas pump. In this and other embodiments, determining to switch the configuration of the first and second heads at block 704 may be based on the measured barometric pressure and capabilities of the multi-head diaphragm gas pump at different barometric pressures. FIG. 8 illustrates an example of how to determine whether to operate the multi-head diaphragm gas pump in parallel or series based on the measured barometric pressure and capabilities of the multi-head diaphragm gas pump, arranged in accordance with at least one embodiment herein.

In the example of FIG. 8, known capabilities of the multi-head diaphragm gas pump at two different barometric pressures may be used to calculate or estimate the capabilities of the multi-head diaphragm gas pump at a third barometric pressure and thereby determine whether to operate the multi-head diaphragm gas pump in parallel or series. The two different barometric pressures may correspond, at least approximately, to two different altitudes, such as sea level and 3,000 meters. In this example, the two different barometric pressures are 14.7 pounds per square inch absolute (psia), which may correspond to sea level, and 10.1 psia, which may correspond to an altitude of 3,000 meters.

The known capabilities of the multi-head diaphragm gas pump may include the maximum achievable pressures of the multi-head diaphragm gas pump with the heads arranged in parallel and in series at each of the two different barometric pressures. The first maximum achievable pressure with the heads in parallel may be referred to as the maximum achievable parallel pressure. The second maximum achievable pressure with the heads in series may be referred to as the maximum achievable series pressure. In some embodiments, the known capabilities of the multi-head diaphragm gas pump at a given one of the two different barometric pressures may be determined in a manufacturing facility or testing facility, e.g., by placing the multi-head diaphragm gas pump in a pressure chamber, pressurizing the chamber to a desired barometric pressure, operating the multi-head diaphragm gas pump in parallel and then in series, measuring the maximum achievable pressure of the multi-head diaphragm gas pump in parallel and series, and saving the two maximum achievable pressure values to memory or other computer-writable storage.

In the example of FIG. 8, at the first barometric pressure of 14.7 psia (e.g., sea level), the capabilities of the multi-head diaphragm gas pump may include a maximum achievable parallel pressure of −11 pounds per square inch gauge (psig), and a maximum achievable series pressure of −13 psig. At the second barometric pressure of 10.1 psia (e.g., 3,000 meters), the capabilities of the multi-head diaphragm gas pump may include a maximum achievable parallel pressure of −6.4 psig, and a maximum achievable series pressure of −8.4 psig. The foregoing data points are shown in the table below:

TABLE 1

| | |
|---|---|
| Barometric | Maximum |
| pressure | achievable pressure |

| | | | |
|---|---|---|---|
| 14.7 | psia (sea level) | −11 | psig parallel |
| 10.1 | psia (3,000 m) | −6.4 | psig parallel |
| 14.7 | psia (sea level) | −13 | psig series |
| 10.1 | psia (3,000 m) | −8.4 | psig series |

FIG. 8 includes a graph with maximum achievable pressure along the x axis and barometric pressure along the y axis. The graph of FIG. 8 includes two curves respectively labeled “parallel” and “series”. Now suppose that a desired vacuum pressure to obtain from the multi-head diaphragm gas pump is −7.5 psig or lower. It can be seen from the series curve that the multi-head diaphragm gas pump can achieve the desired vacuum pressure of −7.5 psig or lower when the heads are configured in series at any of the barometric pressures from 10.1 to 14.7 psia. It can be further seen from the parallel curve that the multi-head diaphragm gas pump can achieve the desired vacuum pressure of −7.5 psig or lower when the heads are configured in parallel at some, but not all, of the barometric pressures in the range from 10.1 to 14.7 psia. In particular, when the heads are configured in parallel, the multi-head diaphragm gas pump can achieve the desired vacuum pressure of −7.5 psig or lower only when the barometric pressure is 11.2 psia or higher. At barometric pressures less than 11.2 psia, however, the multi-head diaphragm gas pump may be unable to achieve the desired vacuum pressure of −7.5 psig or lower.

In view of the foregoing, some embodiments provide a barometric pressure sensor that may be used to measure barometric pressure at a usage location of the multi-head diaphragm gas pump. Given the known capabilities of the multi-head diaphragm gas pump (at two different barometric pressures) as represented by FIG. 8, the measured barometric pressure at the usage location, and a minimum desired vacuum pressure, a control system or other system or device may determine whether the multi-head diaphragm gas pump can achieve the minimum desired vacuum pressure in the parallel mode or the series mode and then set the multi-head diaphragm gas pump in the corresponding mode. For instance, in the example of FIG. 8, if the minimum desired vacuum pressure is −7.5 psig and the measured barometric pressure is greater than 11.2 psia, the multi-head diaphragm gas pump may be set to operate in parallel mode. As another example, if the minimum desired vacuum pressure is −7.5 psig and the measured barometric pressure is less than 11.2 psia, the multi-head diaphragm gas pump may be set to operate in series mode.

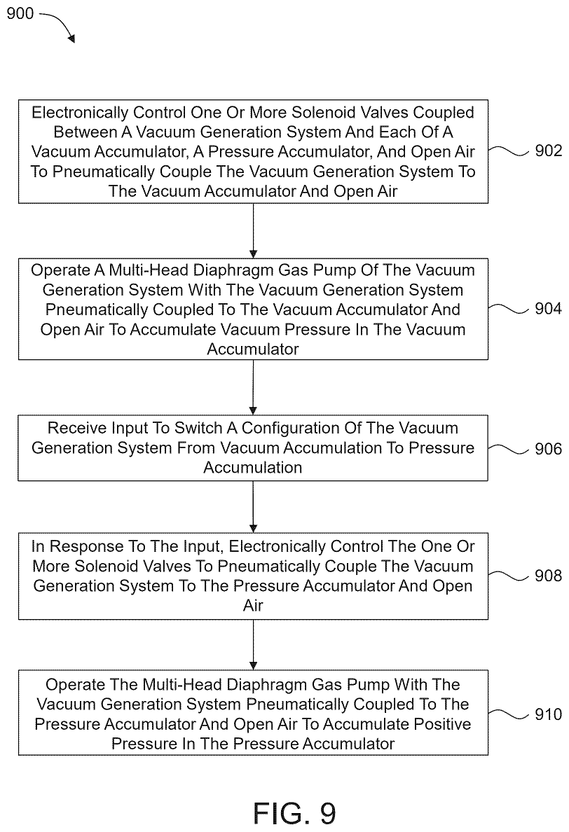

FIG. 9 is a flowchart of another example method 900 to operate a vacuum system for an ophthalmic surgical system, arranged in accordance with at least one embodiment herein. The method 900 may be performed or controlled by any suitable system, apparatus, or device. For example, the method 900 may be implemented, in whole or in part, in the ophthalmic surgical system 100 of FIG. 1, the vacuum system 114 of FIG. 1, the vacuum system 200 of FIG. 2, the vacuum systems of FIGS. 5A-5D, the vacuum systems 600 of FIGS. 6A-6J, or other system or device herein, and/or using the control module 110 of FIG. 1, the control system 312, or other control or computing system herein. Alternatively or additionally, the method 900 may be embodied in computer-readable instructions stored on a computer-readable storage medium, the computer-readable instructions executable by a processor to perform or control performance of the method 900. The method 900 may include one or more of blocks 902, 904, 906, 908, and/or 910. Although illustrated with discrete blocks, the steps and operations associated with one or more blocks of the method 900 may be divided into additional blocks, combined into fewer blocks, or eliminated, depending on the particular implementation.

At block 902, the method 900 may include electronically controlling one or more solenoid valves (e.g., one or more of SVs 512) coupled between a vacuum generation system (e.g., any of the vacuum generation systems 400A-400C, 500A-500C) and each of a vacuum accumulator (e.g., vacuum accumulator 204, 306, 402), a pressure accumulator (e.g., pressure accumulator 510), and open air (e.g., open air 404) to pneumatically couple the vacuum generation system to the vacuum accumulator and open air. Block 902 may be followed by block 904.

At block 904, the method 900 may include operating a diaphragm gas pump (e.g., the pump 406) of the vacuum generation system with the vacuum generation system pneumatically coupled to the vacuum accumulator and open air to accumulate vacuum pressure in the vacuum accumulator. Block 904 may be followed by block 906.

At block 906, the method 900 may include receiving input to switch a configuration of the vacuum generation system from vacuum accumulation to pressure accumulation. The input may be received through an input device coupled to the control system and may come from, e.g., an ophthalmologist or other practitioner performing an ophthalmic surgical procedure using the ophthalmic surgical system that includes the vacuum system. Block 906 may be followed by block 908.

At block 908, and in response to the input, the method 900 may include electronically controlling the one or more solenoid valves to pneumatically couple the vacuum generation system to the pressure accumulator and open air. Block 908 may be followed by block 910.

At block 910, the method 900 may include operating the diaphragm gas pump with the vacuum generation system pneumatically coupled to the pressure accumulator and open air to accumulate positive pressure in the pressure accumulator.

In some embodiments, the method 900 may further include applying the vacuum pressure stored in the vacuum accumulator at a patient interface to suction the patient interface to patient tissue. Alternatively or additionally, the method 900 may further include applying the positive pressure stored in the pressure accumulator at the patient interface to release the patient interface from the patient tissue. Alternatively or additionally, the method 900 may include one or more of the steps or operations described in connection with the method 700 of FIG. 7 or other methods or procedures herein.

An example method to assemble a vacuum system for an ophthalmic surgical system may include one or more of the following steps. The method may include coupling a first inlet port of a first head of a multi-head diaphragm gas pump (or of a first single-head diaphragm gas pump) to a vacuum accumulator. The first head may further include a first outlet port. The method may include coupling a second outlet port of a second head of the multi-head diaphragm gas pump (or of a second single-head diaphragm gas pump) to open air. The second head may further include a second inlet port. The method may include coupling one or more solenoid valves to the vacuum accumulator, the first outlet port of the first head, the second inlet port of the second head, and open air. The one or more solenoid valves may be configured to selectively pneumatically couple the first and second heads in parallel or series. The method may include coupling the vacuum accumulator to a patient interface, the patient interface configured to suction to patient tissue in response to application of vacuum pressure from the vacuum accumulator at the patient interface. The method may include coupling a control system to the one or more solenoid valves. The control system may be configured to electronically control the one or more solenoid valves to selectively pneumatically couple the first and second heads in parallel or series.

In some embodiment, the method to assemble the vacuum system may further include coupling a pressure sensor to the vacuum accumulator and/or to the control system. The control system may be configured to electronically control the one or more solenoid valves to switch the selective pneumatic coupling of the first and second heads between parallel and series based on pressure measurements from the pressure sensor.

Alternatively or additionally, the method to assemble the vacuum system may further include coupling a barometric pressure sensor to the control system. The control system may be configured to electronically control the one or more solenoid valves to switch the selective pneumatic coupling of the first and second heads between parallel and series based on barometric pressure measurements generated by the barometric pressure sensor at a usage location of the vacuum system.

FIG. 10 is a block diagram of an example computing system 1000 suitable for use in implementing some embodiments of the present disclosure. Computing system 1000 may include an interconnect system 1002 that directly or indirectly couples the following devices: memory 1004, one or more central processing units (CPUs) 1006, one or more graphics processing units (GPUs) 1008, a communication interface 1010, I/O ports 1012, input/output components 1014, a power supply 1016, one or more presentation components 1018 (e.g., display(s)), and one or more logic units 1020.

Although the various blocks of FIG. 10 are shown as connected via the interconnect system 1002 with lines, this is not intended to be limiting and is for clarity only. For example, in some embodiments, a presentation component 1018, such as a display device, may be considered an I/O component 1014 (e.g., if the display is a touch screen). As another example, the CPUs 1006 and/or GPUs 1008 may include memory (e.g., the memory 1004 may be representative of a storage device in addition to the memory of the GPUs 1008, the CPUs 1006, and/or other components). In other words, the computing system of FIG. 10 is merely illustrative. Distinction is not made between such categories as “workstation,” “server,” “laptop,” “desktop,” “tablet,” “client device,” “mobile device,” “hand-held device,” “game console,” “electronic control unit (ECU),” “virtual reality system,” “augmented reality system,” and/or other device or system types, as all are contemplated within the scope of the computing system of FIG. 10.

The interconnect system 1002 may represent one or more links or busses, such as an address bus, a data bus, a control bus, or a combination thereof. The interconnect system 1002 may include one or more bus or link types, such as an industry standard architecture (ISA) bus, an extended industry standard architecture (EISA) bus, a video electronics standards association (VESA) bus, a peripheral component interconnect (PCI) bus, a peripheral component interconnect express (PCIe) bus, and/or another type of bus or link. In some embodiments, there are direct connections between components. As an example, the CPU 1006 may be directly connected to the memory 1004. Further, the CPU 1006 may be directly connected to the GPU 1008. Where there is direct, or point-to-point, connection between components, the interconnect system 1002 may include a PCIe link to carry out the connection. In these examples, a PCI bus need not be included in the computing system 1000.

The memory 1004 may include any of a variety of computer-readable media. The computer-readable media may be any available media that may be accessed by the computing system 1000. The computer-readable media may include both volatile and nonvolatile media, and removable and non-removable media. By way of example, and not limitation, the computer-readable media may comprise computer-storage media and communication media.

The computer-storage media may include both volatile and nonvolatile media and/or removable and non-removable media implemented in any method or technology for storage of information such as computer-readable instructions, data structures, program modules, and/or other data types. For example, the memory 1004 may store computer-readable instructions (e.g., that represent a program(s) and/or a program element(s), such as an operating system. Computer-storage media may include, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium that may be used to store the desired information and that may be accessed by computing system 1000. As used herein, computer storage media does not comprise signals per se.

The computer storage media may embody computer-readable instructions, data structures, program modules, and/or other data types in a modulated data signal such as a carrier wave or other transport mechanism and includes any information delivery media. The term “modulated data signal” may refer to a signal that has one or more of its characteristics set or changed in such a manner as to encode information in the signal. By way of example, and not limitation, the computer storage media may include wired media such as a wired network or direct-wired connection, and wireless media such as acoustic, RF, infrared and other wireless media. Combinations of any of the above should also be included within the scope of computer-readable media.

The CPU(s) 1006 may be configured to execute at least some of the computer-readable instructions to control one or more components of the computing system 1000 (or other devices or systems to which the computing system 1000 may be coupled) to perform one or more of the methods and/or processes described herein. The CPU(s) 1006 may each include one or more cores (e.g., one, two, four, eight, twenty-eight, seventy-two, etc.) that are capable of handling a multitude of software threads simultaneously. The CPU(s) 1006 may include any type of processor, and may include different types of processors depending on the type of computing system 1000 implemented (e.g., processors with fewer cores for mobile devices and processors with more cores for servers). For example, depending on the type of computing system 1000, the processor may be an Advanced RISC Machines (ARM) processor implemented using Reduced Instruction Set Computing (RISC) or an x86 processor implemented using Complex Instruction Set Computing (CISC). The computing system 1000 may include one or more CPUs 1006 in addition to one or more microprocessors or supplementary co-processors, such as math co-processors.

In addition to or alternatively from the CPU(s) 1006, the GPU(s) 1008 may be configured to execute at least some of the computer-readable instructions to control one or more components of the computing system 1000 (or other devices or systems to which the computing system 1000 may be coupled) to perform one or more of the methods and/or processes described herein. One or more of the GPU(s) 1008 may be an integrated GPU (e.g., with one or more of the CPU(s) 1006 and/or one or more of the GPU(s) 1008 may be a discrete GPU. In embodiments, one or more of the GPU(s) 1008 may be a coprocessor of one or more of the CPU(s) 1006. The GPU(s) 1008 may be used by the computing system 1000 to render graphics (e.g., 10D graphics) or perform general purpose computations. For example, the GPU(s) 1008 may be used for General-Purpose computing on GPUs (GPGPU). The GPU(s) 1008 may include hundreds or thousands of cores that are capable of handling hundreds or thousands of software threads simultaneously. The GPU(s) 1008 may generate pixel data for output images in response to rendering commands (e.g., rendering commands from the CPU(s) 1006 received via a host interface). The GPU(s) 1008 may include graphics memory, such as display memory, for storing pixel data or any other suitable data, such as GPGPU data. The display memory may be included as part of the memory 1004. The GPU(s) 1008 may include two or more GPUs operating in parallel (e.g., via a link). The link may directly connect the GPUs (e.g., using NVLINK) or may connect the GPUs through a switch (e.g., using NVSwitch). When combined together, each GPU 1008 may generate pixel data or GPGPU data for different portions of an output or for different outputs (e.g., a first GPU for a first image and a second GPU for a second image). Each GPU may include its own memory, or may share memory with other GPUs.

In addition to or alternatively from the CPU(s) 1006 and/or the GPU(s) 1008, the logic unit(s) 1020 may be configured to execute at least some of the computer-readable instructions to control one or more components of the computing system 1000 (or other devices or systems to which the computing system 1000 may be coupled) to perform one or more of the methods and/or processes described herein. In embodiments, the CPU(s) 1006, the GPU(s) 1008, and/or the logic unit(s) 1020 may discretely or jointly perform any combination of the methods, processes and/or portions thereof. One or more of the logic units 1020 may be part of and/or integrated in one or more of the CPU(s) 1006 and/or the GPU(s) 1008 and/or one or more of the logic units 1020 may be discrete components or otherwise external to the CPU(s) 1006 and/or the GPU(s) 1008. In embodiments, one or more of the logic units 1020 may be a coprocessor of one or more of the CPU(s) 1006 and/or one or more of the GPU(s) 1008.

Examples of the logic unit(s) 1020 include one or more processing cores and/or components thereof, such as Tensor Cores (TCs), Tensor Processing Units (TPUs), Pixel Visual Cores (PVCs), Vision Processing Units (VPUs), Graphics Processing Clusters (GPCs), Texture Processing Clusters (TPCs), Streaming Multiprocessors (SMS), Tree Traversal Units (TTUs), Artificial Intelligence Accelerators (AIAs), Deep Learning Accelerators (DLAs), Arithmetic-Logic Units (ALUs), Application-Specific Integrated Circuits (ASICs), Floating Point Units (FPUs), I/O elements, peripheral component interconnect (PCI) or peripheral component interconnect express (PCIe) elements, and/or the like.

The communication interface 1010 may include one or more receivers, transmitters, and/or transceivers that enable the computing system 1000 to communicate with other computing systems via an electronic communication network, including wired and/or wireless communications. The communication interface 1010 may include components and functionality to enable communication over any of a number of different networks, such as wireless networks (e.g., Wi-Fi, Z-Wave, Bluetooth, Bluetooth LE, ZigBee, etc.), wired networks (e.g., communicating over Ethernet or InfiniBand), low-power wide-area networks (e.g., LoRaWAN, SigFox, etc.), and/or the Internet.

The I/O ports 1012 may enable the computing system 1000 to be logically coupled to other devices including the I/O components 1014, the presentation component(s) 1018, and/or other components, some of which may be built into (e.g., integrated in) the computing system 1000. Illustrative I/O components 1014 include a microphone, mouse, keyboard, joystick, game pad, game controller, satellite dish, scanner, printer, wireless device, etc. The I/O components 1014 may provide a natural user interface (NUI) that processes air gestures, voice, or other physiological inputs generated by a user. In some instances, inputs may be transmitted to an appropriate network element for further processing. An NUI may implement any combination of speech recognition, stylus recognition, facial recognition, biometric recognition, gesture recognition both on screen and adjacent to the screen, air gestures, head and eye tracking, and touch recognition (as described in more detail below) associated with a display of the computing system 1000. The computing system 1000 may include depth cameras, such as stereoscopic camera systems, infrared camera systems, RGB camera systems, touchscreen technology, and combinations of these, for gesture detection and recognition. Additionally, the computing system 1000 may include accelerometers or gyroscopes (e.g., as part of an inertia measurement unit (IMU)) that enable detection of motion. In some examples, the output of the accelerometers or gyroscopes may be used by the computing system 1000 to render immersive augmented reality or virtual reality.

The power supply 1016 may include a hard-wired power supply, a battery power supply, or a combination thereof. The power supply 1016 may provide power to the computing system 1000 to enable the components of the computing system 1000 to operate.

The presentation component(s) 1018 may include a display (e.g., a monitor, a touch screen, a television screen, a heads-up-display (HUD), other display types, or a combination thereof), speakers, and/or other presentation components. The presentation component(s) 1018 may receive data from other components (e.g., the GPU(s) 1008, the CPU(s) 1006, etc.), and output the data (e.g., as an image, video, sound, etc.).

Modifications, additions, or omissions may be made to FIG. 10 without departing from the scope of the present disclosure. For example, the computing system 1000 may include more or fewer elements depending on the implementation. Further, the computing system 1000 may be configured to perform any number of operations as compared to those explicitly described.

The disclosure may be described in the general context of computer code or machine-useable instructions, including computer-executable instructions such as program modules, being executed by a computer or other machine, such as a personal data assistant or other handheld device. Generally, program modules including routines, programs, objects, components, data structures, etc., refer to codes that perform particular tasks or implement particular abstract data types. The disclosure may be practiced in a variety of system configurations, including hand-held devices, consumer electronics, general-purpose computers, more specialty computing systems, etc. The disclosure may also be practiced in distributed computing environments where tasks are performed by remote-processing devices that are linked through a communications network.

As used herein, a recitation of “and/or” with respect to two or more elements should be interpreted to mean only one element, or a combination of elements. For example, “element A, element B, and/or element C” may include only element A, only element B, only element C, element A and element B, element A and element C, element B and element C, or elements A, B, and C. In addition, “at least one of element A or element B” may include at least one of element A, at least one of element B, or at least one of element A and at least one of element B. Further, “at least one of element A and element B” may include at least one of element A, at least one of element B, or at least one of element A and at least one of element B. Additionally, use of the term “based on” should not be interpreted as “only based on” or “based only on.” Rather, a first element being “based on” a second element includes instances in which the first element is based on the second element but may also be based on one or more additional elements.

The subject matter of the present disclosure is described with specificity herein to meet statutory requirements. However, the description itself is not intended to limit the scope of this disclosure. Rather, the inventors have contemplated that the claimed subject matter might also be embodied in other ways, to include different steps or combinations of steps similar to the ones described in this document, in conjunction with other present or future technologies. Moreover, although the terms “step” and/or “block” may be used herein to connote different elements of methods employed, the terms should not be interpreted as implying any particular order among or between various steps herein disclosed unless and except when the order of individual steps is explicitly described.

The subject technology of the present disclosure is illustrated, for example, according to various aspects described below. Various examples of aspects of the present disclosure are described as numbered examples (1, 2, 3, etc.) for convenience. These are provided as examples and do not limit the present disclosure. The aspects of the various implementations described herein may be omitted, substituted for aspects of other implementations, or combined with aspects of other implementations unless context dictates otherwise. For example, one or more aspects of example 1 below may be omitted, substituted for one or more aspects of another example (e.g., example 2) or examples, or combined with aspects of another example The following is a non-limiting summary of some example implementations presented herein.

Example 1. A vacuum system for an ophthalmic surgical system, comprising:
a vacuum accumulator;
a patient interface pneumatically coupled to the vacuum accumulator, the patient interface engageable with patient tissue responsive to application of vacuum pressure from the vacuum accumulator through the patient interface to the patient tissue; and
a vacuum generation system coupled to the vacuum accumulator, the vacuum generation system comprising:
multiple diaphragm gas pump heads that include at least a first head and a second head; and
one or more solenoid valves coupled to the first head and the second head and configured to selectively pneumatically couple the first head and the second head in parallel or series.

Example 1A. The vacuum system of any example herein, particularly of example 1, wherein the diaphragm gas pump heads are different heads of the same multi-head diaphragm gas pump.

Example 1B. The vacuum system of any example herein, particularly of example 1, wherein each of the diaphragm gas pump heads is a different single-head diaphragm gas pump.

Example 2. The vacuum system of any example herein, particularly any one of examples 1-1B, wherein the one or more solenoid valves comprise:
two 4/2 solenoid valves;
one 5/3 solenoid valve;
one 5/2 solenoid valve; or
one 4/2 solenoid valve and one 3/2 solenoid valve.

Example 3. The vacuum system of any example herein, particularly any one of examples 1-2, further comprising a pressure sensor configured to measure vacuum pressure in the vacuum accumulator.

Example 4. The vacuum system of any example herein, particularly of example 3, wherein the one or more solenoid valves and the pressure sensor are coupled to a control system configured to:
electronically control the one or more solenoid valves to selectively pneumatically couple the first and second heads in parallel or series;
receive vacuum pressure measurements from the pressure sensor; and
switch the selective pneumatic coupling of the first and second heads from parallel to series in response to at least one of:
the measured vacuum pressure reaching a predetermined threshold; or
the measured vacuum pressure stabilizing.

Example 5. The vacuum system of any example herein, particularly any one of examples claim 1-4, further comprising a barometric pressure sensor coupled to a control system that is also coupled to the one or more solenoid valves, wherein the control system is configured to:
receive a barometric pressure measurement from the barometric pressure sensor; and
electronically control the one or more solenoid valves to selectively pneumatically couple the first and second heads in parallel or series based on a measured barometric pressure and capabilities of the diaphragm gas pump heads at different barometric pressures.

Example 6. The vacuum system of any example herein, particularly any one of examples 1-5, wherein:
the first head includes a first inlet port and a first outlet port;
the second head includes a second inlet port and a second outlet port;
when the first and second heads are coupled in parallel, the first and second inlet ports are both pneumatically coupled to the vacuum accumulator and the first and second outlet ports are both pneumatically coupled to open air; and
when the first and second heads are coupled in series, the first inlet port is pneumatically coupled to the vacuum accumulator, the first outlet port is pneumatically coupled to the second inlet port, and the second outlet port is pneumatically coupled to open air.

Example 7. The vacuum system of any example herein, particularly any one of examples 1-6, further comprising:
a pressure accumulator; and
one or more other solenoid valves coupled between the vacuum generation system and each of the vacuum accumulator, the pressure accumulator, and open air, wherein the one or more other solenoid valves are configured to selectively pneumatically couple the vacuum generation system to:
the vacuum accumulator and open air; or
the pressure accumulator and open air.

Example 8. A method to operate a vacuum system for an ophthalmic surgical system, the method comprising:
electronically controlling one or more solenoid valves coupled between first and second diaphragm gas pump heads of one or more pumps to pneumatically couple the first and second heads in parallel;
determining to switch a configuration of the first and second heads;
in response to the determination, electronically controlling the one or more solenoid valves to pneumatically couple the first and second heads in series;
operating the first and second heads with the first and second heads pneumatically coupled in parallel or series to accumulate vacuum pressure in a vacuum accumulator pneumatically coupled to the first and second heads; and
applying the vacuum pressure stored in the vacuum accumulator at a patient interface to suction the patient interface to patient tissue.

Example 8A. The method of any example herein, particularly of example 8, wherein the first and second heads are different heads of the same multi-head diaphragm gas pump.

Example 8B. The method of any example herein, particularly of example 8, wherein each of the first and second heads is a different single-head diaphragm gas pump.

Example 9. The method of any example herein, particularly any one of examples 8-8B, wherein:
operating the first and second heads with the first and second heads pneumatically coupled in parallel or series includes operating the first and second heads with the first and second heads pneumatically coupled in parallel after electronically controlling the one or more solenoid valves coupled between the first and second heads to pneumatically couple the first and second heads in parallel and prior to determining to switch the configuration of the first and second heads;
the method further comprises monitoring vacuum pressure in the vacuum accumulator, while operating the first and second heads with the first and second heads pneumatically coupled in parallel, prior to determining to switch the configuration of the first and second heads; and
determining to switch the configuration of the first and second heads is based on the monitored vacuum pressure in the vacuum accumulator reaching a predetermined threshold.

Example 10. The method of any example herein, particularly any one of examples 8-9, wherein:
operating the first and second heads with the first and second heads pneumatically coupled in parallel or series further includes operating the first and second heads with the first and second heads pneumatically coupled in series after electronically controlling the one or more solenoid valves to pneumatically couple the first and second heads in series;
the method further comprises continuing to monitor vacuum pressure in the vacuum accumulator while operating the first and second heads with the first and second heads pneumatically coupled in series; and
turning off the first and second heads in response to the monitored vacuum pressure in the vacuum accumulator reaching a second predetermined threshold.

Example 11. The method of any example herein, particularly any one of examples 8-8B, further comprising, during an ophthalmic surgical procedure:
monitoring vacuum pressure in the vacuum accumulator while operating the first and second heads with the first and second heads pneumatically coupled in parallel;
determining based on the monitored vacuum pressure in the vacuum accumulator that vacuum pressure in the vacuum accumulator has stabilized with the first and second heads pneumatically coupled in parallel, wherein determining to switch the configuration of the first and second heads is based on determining that vacuum pressure in the vacuum accumulator has stabilized with the first and second heads pneumatically coupled in parallel;
continuing to monitor vacuum pressure in the vacuum accumulator while operating the first and second heads with the first and second heads pneumatically coupled in series;
determining based on the monitored vacuum pressure in the vacuum accumulator that vacuum pressure in the vacuum accumulator has stabilized with the first and second heads pneumatically coupled in series; and
turning off the first and second heads in response to determining that vacuum pressure in the vacuum accumulator has stabilized with the first and second heads pneumatically coupled in series.

Example 12. The method of any example herein, particularly any one of examples 8-11, further comprising electronically controlling one or more other solenoid valves coupled between the vacuum generation system and each of the vacuum accumulator, a pressure accumulator, and open air to selectively pneumatically couple the vacuum generation system to:
the vacuum accumulator and open air; or
the pressure accumulator and open air.

Example 13. The method of any example herein, particularly any one of examples 8-12, further comprising receiving measured barometric pressure of a usage location of the first and second heads, wherein determining to switch the configuration of the first and second heads is based on the measured barometric pressure and capabilities of the first and second heads at different barometric pressures.

Example 14. A method to assemble a vacuum system for an ophthalmic surgical system, the method comprising:
coupling a first inlet port of a first diaphragm gas pump head to a vacuum accumulator; the first head further comprising a first outlet port;
coupling a second outlet port of a second diaphragm gas pump head to open air; the second head further comprising a second inlet port;
coupling one or more solenoid valves to the vacuum accumulator, the first outlet port of the first head, the second inlet port of the second head, and open air, the one or more solenoid valves configured to selectively pneumatically couple the first and second heads in parallel or series;
coupling the vacuum accumulator to a patient interface, the patient interface configured to suction to patient tissue in response to application of vacuum pressure from the vacuum accumulator at the patient interface; and
coupling a control system to the one or more solenoid valves, the control system configured to electronically control the one or more solenoid valves to selectively pneumatically couple the first and second heads in parallel or series.

Example 14A. The method of any example herein, particularly of example 14, wherein the first and second heads are different heads of the same multi-head diaphragm gas pump.

Example 14B. The method of any example herein, particularly of example 14, wherein each of the first and second heads is a different single-head diaphragm gas pump.

Example 15. The method of any example herein, particularly any one of examples 14-14B, further comprising coupling a pressure sensor to the vacuum accumulator and to the control system, wherein the control system is configured to electronically control the one or more solenoid valves to switch the selective pneumatic coupling of the first and second heads between parallel and series based on pressure measurements from the pressure sensor.

Example 16. The method of any example herein, particularly any one of examples 14-15, further comprising coupling a barometric pressure sensor to the control system, wherein the control system is configured to electronically control the one or more solenoid valves to switch the selective pneumatic coupling of the first and second heads between parallel and series based on barometric pressure measurements generated by the barometric pressure sensor at a usage location of the vacuum system.

Example 17. A vacuum system for an ophthalmic surgical system, comprising:
a vacuum accumulator;
a pressure accumulator;
a patient interface pneumatically coupled to the vacuum accumulator, the patient interface engageable with patient tissue responsive to application of vacuum pressure from the vacuum accumulator through the patient interface to the patient tissue;
a vacuum generation system, comprising multiple diaphragm gas pump heads that include at least a first head and a second head, the first head including a first inlet port and a first outlet port and the second head including a second inlet port and a second outlet port; and
one or more solenoid valves coupled between the vacuum generation system and each of the vacuum accumulator, the pressure accumulator, and open air, the one or more solenoid valves configured to selectively pneumatically couple the vacuum generation system to:
the vacuum accumulator and open air; or
the pressure accumulator and open air.

Example 18. The vacuum system of any example herein, particularly of example 17, wherein the one or more solenoid valves comprise:
two 4/2 solenoid valves;
one 5/3 solenoid valve;
one 5/2 solenoid valve; or
one 4/2 solenoid valve and one 3/2 solenoid valve.

Example 19. The vacuum system of any example herein, particularly any one of examples 17-18, further comprising one or more other solenoid valves coupled to the first head and the second and configured to selectively pneumatically couple the first and second heads in parallel or series.

Example 20. The vacuum system of any example herein, particularly any one of examples 17-19, wherein the one or more solenoid valves includes one of:
a single solenoid valve having a first port coupled to open air, a second port coupled to a first inlet port of the first head of the vacuum generation system, a third port coupled to the vacuum accumulator, a fourth port coupled to a second outlet port of the second head of the vacuum generation system, and a fifth port coupled to the pressure accumulator;
a first solenoid valve having a first port coupled to a second outlet port of the second head of the vacuum generation system, a second port, a third port coupled to a first inlet port of the first head of the vacuum generation system, and a fourth port coupled to open air, and a second solenoid valve having a first port coupled to the second port of the first solenoid valve, a second port coupled to the pressure accumulator, a third port, and a fourth port coupled to the vacuum accumulator; or
a third solenoid valve having a first port coupled to a second outlet port of the second head of the vacuum generation system, a second port, a third port coupled to a first inlet port of the first head of the vacuum generation system, and a fourth port coupled to open air, and a fourth solenoid valve having a first port coupled to the vacuum accumulator, a second port coupled to the second port of the third solenoid valve, and a third port coupled to the pressure accumulator.

Example 21. A method to operate a vacuum system for an ophthalmic surgical system, the method comprising:
electronically controlling one or more solenoid valves coupled between a vacuum generation system and each of a vacuum accumulator, a pressure accumulator, and open air to pneumatically couple the vacuum generation system to the vacuum accumulator and open air;
operating multiple diaphragm gas pump heads of the vacuum generation system with the vacuum generation system pneumatically coupled to the vacuum accumulator and open air to accumulate vacuum pressure in the vacuum accumulator;
receiving input to switch a configuration of the vacuum generation system from vacuum accumulation to pressure accumulation;
in response to the input, electronically controlling the one or more solenoid valves to pneumatically couple the vacuum generation system to the pressure accumulator and open air; and
operating the multiple diaphragm gas pump heads with the vacuum generation system pneumatically coupled to the pressure accumulator and open air to accumulate positive pressure in the pressure accumulator.

Example 22. The method of any example herein, particularly of example 21, further comprising, applying the vacuum pressure stored in the vacuum accumulator at a patient interface to suction the patient interface to patient tissue.

Example 23. The method of any example herein, particularly of example 21, further comprising, applying the positive pressure stored in the pressure accumulator at the patient interface to release the patient interface from the patient tissue.

Example 24. The method of any example herein, particularly of example 21, further comprising, applying the vacuum pressure stored in the vacuum accumulator at a laser head to suction the laser head to a patient interface.

What is claimed is:

1. A vacuum system for an ophthalmic surgical system, comprising:
a vacuum accumulator;
a patient interface pneumatically coupled to the vacuum accumulator, the patient interface engageable with patient tissue responsive to application of vacuum pressure from the vacuum accumulator through the patient interface to the patient tissue; and
a vacuum generation system coupled to the vacuum accumulator, the vacuum generation system comprising:
multiple diaphragm gas pump heads, including at least a first head and a second head; and
one or more solenoid valves coupled to the first head and the second head and configured to selectively pneumatically couple the first head and the second head in parallel or series.

2. The vacuum system of claim 1, wherein the one or more solenoid valves comprise:
two 4/2 solenoid valves;
one 5/3 solenoid valve;
one 5/2 solenoid valve; or
one 4/2 solenoid valve and one 3/2 solenoid valve.

3. The vacuum system of claim 1, further comprising a pressure sensor configured to measure vacuum pressure in the vacuum accumulator.

4. The vacuum system of claim 3, wherein the one or more solenoid valves and the pressure sensor are coupled to a control system configured to:
electronically control the one or more solenoid valves to selectively pneumatically couple the first and second heads in parallel or series;
receive vacuum pressure measurements from the pressure sensor; and
switch the selective pneumatic coupling of the first and second heads from parallel to series in response to at least one of:
the measured vacuum pressure reaching a predetermined threshold; or
the measured vacuum pressure stabilizing.

5. The vacuum system of claim 1, further comprising a barometric pressure sensor coupled to a control system that is also coupled to the one or more solenoid valves, wherein the control system is configured to:
receive a barometric pressure measurement from the barometric pressure sensor; and
electronically control the one or more solenoid valves to selectively pneumatically couple the first and second heads in parallel or series based on a measured barometric pressure and capabilities of the diaphragm gas pump heads at different barometric pressures.

6. The vacuum system of claim 1, wherein:
the first head includes a first inlet port and a first outlet port;
the second head includes a second inlet port and a second outlet port;
when the first and second heads are coupled in parallel, the first and second inlet ports are both pneumatically coupled to the vacuum accumulator and the first and second outlet ports are both pneumatically coupled to open air; and
when the first and second heads are coupled in series, the first inlet port is pneumatically coupled to the vacuum accumulator, the first outlet port is pneumatically coupled to the second inlet port, and the second outlet port is pneumatically coupled to open air.

7. The vacuum system of claim 1, further comprising:
a pressure accumulator; and
one or more other solenoid valves coupled between the vacuum generation system and each of the vacuum accumulator, the pressure accumulator, and open air, wherein the one or more other solenoid valves are configured to selectively pneumatically couple the vacuum generation system to:
the vacuum accumulator and open air; or
the pressure accumulator and open air.

8. A method to operate a vacuum system for an ophthalmic surgical system, the method comprising:
electronically controlling one or more solenoid valves coupled between first and second diaphragm gas pump heads of one or more pumps of a vacuum generation system to pneumatically couple the first and second heads in parallel;
determining to switch a configuration of the first and second heads;
in response to the determination, electronically controlling the one or more solenoid valves to pneumatically couple the first and second heads in series;
operating the first and second heads with the first and second heads pneumatically coupled in parallel or series to accumulate vacuum pressure in a vacuum accumulator pneumatically coupled to the first and second heads; and
applying the vacuum pressure stored in the vacuum accumulator at a patient interface to suction the patient interface to patient tissue.

9. The method of claim 8, wherein:
operating the first and second heads with the first and second heads pneumatically coupled in parallel or series includes operating the first and second heads with the first and second heads pneumatically coupled in parallel after electronically controlling the one or more solenoid valves coupled between the first and second heads to pneumatically couple the first and second heads in parallel and prior to determining to switch the configuration of the first and second heads;
the method further comprises monitoring vacuum pressure in the vacuum accumulator, while operating the first and second heads with the first and second heads pneumatically coupled in parallel, prior to determining to switch the configuration of the first and second heads; and
determining to switch the configuration of the first and second heads is based on the monitored vacuum pressure in the vacuum accumulator reaching a predetermined threshold.

10. The method of claim 9, wherein:
operating the first and second heads with the first and second heads pneumatically coupled in parallel or series further includes operating the first and second heads with the first and second heads pneumatically coupled in series after electronically controlling the one or more solenoid valves to pneumatically couple the first and second heads in series;
the method further comprises continuing to monitor vacuum pressure in the vacuum accumulator while operating the first and second heads with the first and second heads pneumatically coupled in series; and
turning off the first and second heads in response to the monitored vacuum pressure in the vacuum accumulator reaching a second predetermined threshold.

11. The method of claim 8, further comprising, during an ophthalmic surgical procedure:
monitoring vacuum pressure in the vacuum accumulator while operating the first and second heads with the first and second heads pneumatically coupled in parallel;
determining based on the monitored vacuum pressure in the vacuum accumulator that vacuum pressure in the vacuum accumulator has stabilized with the first and second heads pneumatically coupled in parallel, wherein determining to switch the configuration of the first and second heads is based on determining that vacuum pressure in the vacuum accumulator has stabilized with the first and second heads pneumatically coupled in parallel;
continuing to monitor vacuum pressure in the vacuum accumulator while operating the first and second heads with the first and second heads pneumatically coupled in series;
determining based on the monitored vacuum pressure in the vacuum accumulator that vacuum pressure in the vacuum accumulator has stabilized with the first and second heads pneumatically coupled in series; and
turning off the first and second heads in response to determining that vacuum pressure in the vacuum accumulator has stabilized with the first and second heads pneumatically coupled in series.

12. The method of claim 8, further comprising electronically controlling one or more other solenoid valves coupled between the vacuum generation system and each of the vacuum accumulator, a pressure accumulator, and open air to selectively pneumatically couple the vacuum generation system to:
the vacuum accumulator and open air; or
the pressure accumulator and open air.

13. The method of claim 8, further comprising receiving measured barometric pressure of a usage location of the first and second heads, wherein determining to switch the configuration of the first and second heads is based on the measured barometric pressure and capabilities of the first and second heads at different barometric pressures.

14. A vacuum system for an ophthalmic surgical system, comprising:
a vacuum accumulator;
a pressure accumulator;
a patient interface pneumatically coupled to the vacuum accumulator, the patient interface engageable with patient tissue responsive to application of vacuum pressure from the vacuum accumulator through the patient interface to the patient tissue;
a vacuum generation system, comprising multiple diaphragm gas pump heads that include at least a first head and a second head; and
one or more solenoid valves coupled between the vacuum generation system and each of the vacuum accumulator, the pressure accumulator, and open air, the one or more solenoid valves configured to selectively pneumatically couple the vacuum generation system to:
the vacuum accumulator and open air; or
the pressure accumulator and open air.

15. The vacuum system of claim 14, wherein the one or more solenoid valves comprise:
two 4/2 solenoid valves;
one 5/3 solenoid valve;
one 5/2 solenoid valve; or
one 4/2 solenoid valve and one 3/2 solenoid valve.

16. The vacuum system of claim 14, further comprising one or more other solenoid valves coupled to the first head and the second and configured to selectively pneumatically couple the first head and the second head in parallel or series.

17. The vacuum system of claim 14, wherein the one or more solenoid valves includes one of:
a single solenoid valve having a first port coupled to open air, a second port coupled to a first inlet port of the first head of the vacuum generation system, a third port coupled to the vacuum accumulator, a fourth port coupled to a second outlet port of the second head of the vacuum generation system, and a fifth port coupled to the pressure accumulator;
a first solenoid valve having a first port coupled to a second outlet port of the second head of the vacuum generation system, a second port, a third port coupled to a first inlet port of the first head of the vacuum generation system, and a fourth port coupled to open air, and a second solenoid valve having a first port coupled to the second port of the first solenoid valve, a second port coupled to the pressure accumulator, a third port, and a fourth port coupled to the vacuum accumulator; or
a third solenoid valve having a first port coupled to a second outlet port of the second head of the vacuum generation system, a second port, a third port coupled to a first inlet port of the first head of the vacuum generation system, and a fourth port coupled to open air, and a fourth solenoid valve having a first port coupled to the vacuum accumulator, a second port coupled to the second port of the third solenoid valve, and a third port coupled to the pressure accumulator.

18. A method to operate a vacuum system for an ophthalmic surgical system, the method comprising:
electronically controlling one or more solenoid valves coupled between a vacuum generation system and each of a vacuum accumulator, a pressure accumulator, and open air to pneumatically couple the vacuum generation system to the vacuum accumulator and open air;
operating multiple diaphragm gas pump heads of the vacuum generation system with the vacuum generation system pneumatically coupled to the vacuum accumulator and open air to accumulate vacuum pressure in the vacuum accumulator;
receiving input to switch a configuration of the vacuum generation system from vacuum accumulation to pressure accumulation;
in response to the input, electronically controlling the one or more solenoid valves to pneumatically couple the vacuum generation system to the pressure accumulator and open air; and
operating the multiple diaphragm gas pump heads with the vacuum generation system pneumatically coupled to the pressure accumulator and open air to accumulate positive pressure in the pressure accumulator.

19. The method of claim 18, further comprising, applying the vacuum pressure stored in the vacuum accumulator at a patient interface to suction the patient interface to patient tissue.

20. The method of claim 18, further comprising, applying the positive pressure stored in the pressure accumulator at a patient interface to release the patient interface from patient tissue.
